(19) United States
(12) Patent Application Publication
(10) Pub. No.: US 2007/0059316 A1
(43) Pub. Date: Mar. 15, 2007

(54) Singlet oxygen photosensitizers activated by target binding enhancing the selectivity of targeted pdt agents

(76) Inventors: Alexander J. Pallenberg, Snoqualmie, WA (US); James C. Chen, Snoqualmie, WA (US)

Correspondence Address: SEED INTELLECTUAL PROPERTY LAW GROUP PLLC, 701 FIFTH AVE, SUITE 5400, SEATTLE, WA, 98104, US

(21) Appl. No.: 10/517,569

(22) PCT Filed: Sep. 23, 2004
(86) PCT No.: PCT/US04/31342

§ 371 Date: Oct. 11, 2006

Related U.S. Application Data
(60) Provisional application No. 60/506,378, filed on Sep. 23, 2003.

(51) Int. Cl. A61K 39/395; A61K 38/40; A61K 31/58; A61K 31/715; C07D 487/22; C07K 16/46; C07K 14/80

(52) U.S. Cl. 424/178.1; 514/6; 514/176; 514/54; 514/185; 514/410; 530/391.1; 530/400; 536/17.4; 536/18.7; 540/2; 540/145

(57) ABSTRACT

Provided herein are conjugates, kits, articles of manufacture and methods for detection, diagnosis and treatment by photodynamic therapy of certain undesired biological substrate targets, including, but not limited to bacteria, viruses, and other pathogenic microorganisms, tumors and hyperproliferative tissue. In particular, the conjugates provided include a fluorophore or photosensitizer linked to a targeting moiety and a quenching agent in such a way that activation of the fluorophore or the photosensitizer is quenched unless the targeting moiety is bound to a target, whereupon the quenching agent dissociates or moves away from the photosensitizer, enabling activation of the photosensitizer upon irradiation with light of a suitable wavelength.

### RELATED APPLICATIONS

Benefit of priority is claimed under 35 U.S.C. §119(e) to U.S. provisional patent application No. 60/506,378, filed Sep. 23, 2003, to Pallenberg et al., entitled “SINGLET OXYGEN PHOTOSENSITIZERS ACTIVATED BY TARGET BINDING ENHANCING THE SELECTIVITY OF TARGETED PDT AGENTS.” The above-referenced application is incorporated herein by reference in its entirety.

### TECHNICAL FIELD

Provided herein are compositions and methods of making compositions for enhancing the action of fluorescence detection or photodynamic therapy for the purpose of detecting or destroying tumors, hyperproliferative tissue, or other undesired biological structures.

### BACKGROUND

Photodynamic therapy (“PDT”) is a treatment method for the destruction of tumors and hyperproliferative tissue. PDT is based on the accumulation of a photosensitizer in malignant tissue and hyperproliferative tissue after the administration of the photosensitizer. Subsequent illumination with light of an appropriate wavelength creates a photochemical reaction, a so-called photodynamic effect (for example, a photochemical reaction producing singlet oxygen) that results in tumor destruction.

Photodynamic therapy is effective in destroying abnormal tissue such as cancer cells. In this therapy, a photoreactive agent having a characteristic light absorption waveband is first administered to the patient, typically either orally or by injection. Abnormal tissue or hyperproliferating tissue in the body is known to selectively absorb certain photoreactive agents to a much greater extent than normal tissue, e.g., tumors of the pancreas and colon may absorb two to three times the volume of these agents, compared to normal tissue.

Photosensitizers, such as porphyrins and related tetrapyrrolic compounds, tend to localize in abnormal tissue, including malignant tumors and other hyperproliferative tissue, such as hyperproliferative blood vessels, at much higher concentrations than in normal tissues, so they are useful as a tool for the treatment of various type of cancers and other hyperproliferative tissue by photodynamic therapy (PDT) (T. J. Dougherty, C. J. Gomer, B. W. Henderson, G. Jori, D. Kessel, M. Korbelik, J. Moan, Q. Peng, J. Natl. Cancer Inst. 90: 889 (1998), incorporated here by reference ). Most of the porphyrin-based photosensitizers approved for the treatment of tumors and hyperproliferative tissue clear slowly from normal tissue, so patients must avoid exposure to sunlight for a significant time after treatment in order to minimize unwanted activity of the photosensitizer in non-target tissue. Although photodynamic therapy is effective, there are undesirable side-effects resulting from, for example, the required dosage and inadvertent activation in non-target tissues. Thus, there is a need to improve targeting and delivery of this therapy. Therefore, among the objects herein, it is an object to provide methods and compositions for targeting and delivery of photodynamic therapy.

### SUMMARY

Provided are methods and conjugates for targeting and delivery of photodynamic therapy and for imaging. The conjugates are targeted and are designed so that they are inactive until interacting with a target, such as a targeted tissue or cell. The conjugates are used in methods of photodynamic therapy and imaging and any method in which targeted delivery of a light-generating agent is employed. Also provided are the methods in which the conjugates are used or administered, such as probes in microscopy, enzymology, clinical chemistry, molecular biology and medicine and other such applications. The conjugates also are therapeutic agents in modalities, such as photodynamic therapy and as diagnostic agents in imaging methods, such as fluorescence immunoassays, fluorescent in vivo imaging and magnetic resonance imaging.

The conjugates provided herein include a donor moiety, such as a fluorophore or a photosensitizer, an acceptor moiety, such as a quenching agent, and a targeting moiety. The conjugates contain a donor, such as a fluorophore, photosensitizer and other such agent, linked to a targeting moiety and to an acceptor moiety, such as a quenching agent, in such a way that activation of the donor, such as the fluorophore or the photosensitizer, is quenched unless and until the targeting moiety is bound to a target. Upon binding to a target, the acceptor moiety, such as the quenching agent, dissociates or moves away from the donor agent, such as the photosensitizer, whereby the donor is activated or active. For example, for conjugates containing a photosensitizer, binding to the target results in activation of the photosensitizer upon irradiation with light of a suitable wavelength.

Also provided are conjugates that include a photosensitizer and a quenching agent, where the photosensitizer and the quenching agent include a linking component to link with an amino or hydroxy fatty acid or sulfonic acid using ester, amide, or sulfonamide linkages.

Also provided are conjugates that include a photosensitizer and a quenching agent, where the photosensitizer and the quenching agent include an oligonucleotide as a linking component, where the oligonucleotide includes a specific sequence for binding to a desired target, along with at least one pair of mutually complementary regions that cause it to adopt a conformation, in the absence of the target, in which the quenching agent is sufficiently near to the photosensitizer to render the photosensitizer inactive and where binding of the target-specific sequence to the target disrupts the conformation, allowing the photosensitizer to become active upon illumination with light of an appropriate wavelength.

Also provided are conjugates that include a photosensitizer and a quenching agent, where the photosensitizer comprises a porphyrin or porphyrin derivative tetrapyrrole and bears a physiologically acceptable metal atom in its central coordination cavity and one or more suitable functional groups are located on or near the quenching agent that efficiently coordinate to the axial position of the metal coordinated within the photosensitizer; and the targeting moiety is located in such a way that the presence of the target disrupts the association of the axial ligand to the metal, releasing the quenching agent and rendering the fluorophore or photosensitizing agent active.

Also provided are conjugates that include a photosensitizing agent linked to a targeting moiety and a quenching agent, where the photosensitizing agent and the quenching agent are positioned in an interaction-permissive energy transfer conformation in such a way that activation of the photosensitizer is quenched unless the targeting moiety is bound to a target; and the targeting moiety is positioned so that upon binding of the targeting moiety to a target, the quenching agent is displaced from the interaction-permissive energy transfer conformation with the photosensitizer, enabling activation of the photosensitizer upon irradiation with light of a suitable wavelength.

Also provided are conjugates that include a tetrapyrrole or tetrapyrrole derivative photosensitizer that includes a physiologically acceptable metal atom in its central coordination cavity, a quenching agent including one or more suitable functional groups that coordinate to the axial position of the metal coordinated within the photosensitizer and that position the quenching agent in an energy transfer conformation with the photosensitizer so that activation of the photosensitizer is quenched; and a targeting moiety, wherein binding of the targeting moiety to a target disrupts the association of the axial ligand of the quenching agent to the metal, releasing the quenching agent and enabling activation of the photosensitizer upon irradiation with light of a suitable wavelength.

Also provided are methods for detecting target tissue or target compositions. Further provided herein are methods for photodynamic therapy using the conjugates provided herein. Also provided herein are methods for detecting hyperproliferative tissue using the conjugates provided herein.

Also provided is the use of the conjugates provided herein for the treatment of target compositions or target tissue, including hyperproliferative tissue and neovascular tissue. Provided herein also are methods for detecting the presence of hyperproliferative tissue in a subject. Also provided are methods of diagnosing hyperproliferative disorders in a patient. Further provided is a method of diagnosing an infecting agent in a patient.

Provided herein is also a method of generating an image of a target tissue in a subject. Further provided is a kit to treat hyperproliferative disorders. Also provided is a kit to label specific tissues for diagnostic analysis. Further provided is a combination, including any of the conjugates provided herein and a light source.

### BRIEF DESCRIPTION OF THE DRAWINGS

FIG. 1 is a schematic representation of binding reaction of a targeted photosensitizer to a target.

FIG. 2 illustrates a photosensitizer associated with a linking agent.

FIG. 3 illustrates a binding-activated photosensitizer.

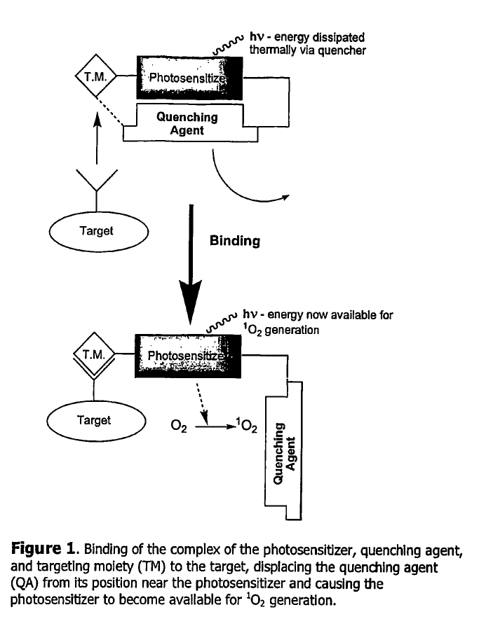

### DETAILED DESCRIPTION

A. Definitions

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, the term “photodynamic therapy” denotes a process whereby light of a specific wavelength is directed to tissues undergoing treatment or investigation that have been rendered photosensitive through the administration of a photoreactive or photosensitizing agent. The objective may be either diagnostic, where the wavelength of light is selected to cause the photoreactive agent to fluoresce, thus yielding information about the tissue without damaging the tissue, or therapeutic, where the wavelength of light delivered to the target tissue under treatment causes the photoreactive agent to undergo a photochemical interaction with oxygen in the tissue under treatment that yields high energy species, such as singlet oxygen, causing local tissue lysing or destruction. The method of van Lier (Photobiological Techniques 216: 85-98 (Valenzo et al., eds. 1991)) can be used to confirm the ability of any given composition to generate singlet oxygen effectively, thus making it a good candidate for use in photodynamic therapy.

As used herein, the term “photosensitizer” or “photosensitizing agent” denotes a chemical compound that upon exposure to photoactivating light is activated, converting the photosensitizing agent itself, or some other species, into a cytotoxic form, whereby target cells are killed or their proliferative potential diminished. Thus, photosensitizing agents may exert their effects by a variety of mechanisms, directly or indirectly. For example, certain photosensitizing agents become directly toxic when activated by light, whereas others act to generate toxic species, e.g. oxidizing agents such as singlet oxygen or oxygen-derived free radicals, which are extremely destructive to cellular material and biomolecules such as lipids, proteins and nucleic acids. Psoralens are exemplary of directly acting photosensitizers; upon exposure to light they form adducts and cross-links between the two strands of DNA molecules, thereby inhibiting DNA synthesis. Porphyrins are exemplary of photosensitizing agents that act indirectly by generation of toxic oxygen species. Virtually any chemical compound that, upon exposure to photoactivating light, is converted into or gives rise to a cytotoxic form may be used in this invention. Generally, the chemical compound is nontoxic to the animal to which it is administered or is capable of being formulated in a nontoxic composition, and the chemical compound in its photodegraded form is also nontoxic. A listing of representative photosensitive chemicals may be found in Kreimer-Bimbaurn, Sem. Hematol. 26:157-73, 1989.

Photosensitive compounds include, but are not limited to, chlorins, bacteriochlorins, phthalocyanines, porphyrins, purpurinimides, pheophorbides, pyropheophorbides, merocyanines, psoralens, benzoporphyrin derivatives (BPD), talaporfin sodium and porfimer sodium and pro-drugs such as deltaaminolevulinic acid, which can produce drugs such as protoporphyrin. Other compounds include indocyanine green; methylene blue; toluidine blue; texaphyrins; and any other agent that absorbs light in a range of 400 nm-1200 nm.

As used herein, the term “tetrapyrrole” denotes a macrocyclic compound containing four pyrrole rings, having the general structure: 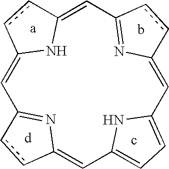
where the dashed line indicates that the indicated bond may be saturated or unsaturated, and where any atom of the ring may be substituted or unsubstituted.

As used herein, the term “porphyrin” refers to a cyclic structure typically composed of four pyrrole rings, and refers to a porphyrin or porphyrin derivative. Such derivatives include porphyrins with extra rings ortho-fused, or ortho-perifused, to the porphyrin nucleus, porphyrins having a replacement of one or more carbon atoms of the porphyrin ring by an atom of another element (skeletal replacement), derivatives having a replacement of a nitrogen atom of the porphyrin ring by an atom of another element (skeletal replacement of nitrogen), derivatives having substituents other than hydrogen located at the peripheral (meso-, □-) or core atoms of the porphyrin, derivatives with saturation of one or more bonds of the porphyrin (hydroporphyrins, e.g., chlorins, bacteriochlorins, isobacteriochlorins, corphins, pyrrocorphins, etc.), derivatives obtained by coordination of one or more metals to one or more porphyrin atoms (metalloporphyrins), derivatives having one or more atoms, including pyrrolic and pyrromethenyl units, inserted in the porphyrin ring (expanded porphyrins), derivatives having one or more groups removed from the porphyrin ring (contracted porphyrins, e.g., corrin, corrole) and combinations of the foregoing derivatives (e.g phthalocyanines, porphyrazines, naphthalocyanines, subphthalocyanines, and porphyrin isomers).

As used herein, “chlorin” refers to a class of porphyrin derivatives having a cyclic structure typically composed of four pyrrole rings having one partially saturated pyrrole ring, such as the basic chromophore of chlorophyll.

As used herein, “bacteriochlorin” refers to a class of porphyrin derivatives having a cyclic structure typically composed of four pyrrole rings having two partially saturated non-adjacent (i.e., trans) pyrrole rings, and “isobacteriochlorin” includes those porphyrin derivatives having a cyclic structure typically composed of four pyrrole rings having two partially saturated adjacent (i.e., cis) pyrrole rings.

As used herein, a “molecule” refers to any molecular entity and includes, but is not limited to, small organic molecules, biopolymers, biomolecules, macromolecules or components or precursors thereof, such as peptides, proteins, organic compounds, oligonucleotides or monomeric units of the peptides, organics, nucleic acids and other macromolecules. A monomeric unit refers to one of the constituents from which the resulting compound is built. Thus, monomeric units include, nucleotides, amino acids, and pharmacophores from which small organic molecules are synthesized.

As used herein, a “biomolecule” is any molecule that occurs in nature, or derivatives thereof. Biomolecules include biopolymers and macromolecules and all molecules that can be isolated from living organisms and viruses, including, but are not limited to, cells, tissues, prions, animals, plants, viruses, bacteria, prions and other organisms. Biomolecules also include, but are not limited to oligonucleotides, oligonucleosides, proteins, peptides, amino acids, lipids, steroids, peptide nucleic acids (PNAs), oligosaccharides and monosaccharides, organic molecules, such as enzyme cofactors, metal complexes, such as heme, iron sulfur clusters, porphyrins and metal complexes thereof, metals, such as copper, molybedenum, zinc and others.

As used herein, “macromolecule” refers to any molecule having a molecular weight from the hundreds up to the millions. Macromolecules include, but are not limited to, peptides, proteins, nucleotides, nucleic acids, carbohydrates, and other such molecules that are generally synthesized by biological organisms, but can be prepared synthetically or using recombinant molecular biology methods.

As used herein, “biopolymer” refers to a biomolecule, including macromolecules, composed of two or more monomeric subunits, or derivatives thereof, which are linked by a bond or a macromolecule. A biopolymer can be, for example, a polynucleotide, a polypeptide, a carbohydrate, or a lipid, or derivatives or combinations thereof, for example, a nucleic acid molecule containing a peptide nucleic acid portion or a glycoprotein.

As used herein, a “donor molecule” refers to a chemical or biological compound that is capable of contributing or transferring energy from itself to another molecule. The energy that is transferred can include, but is not limited to, an electron, a photon, or fluorescence resonance energy.

As used herein, an “acceptor molecule” refers to a chemical or biological compound that is capable of receiving or accepting energy from another molecule. The energy that is transferred can include, but is not limited to, an electron, a photon, or fluorescence resonance energy. Acceptance of energy by the acceptor molecule from the donor molecule by energy transfer mechanisms results in apparent diminished energy of the donor molecule. Energy transfer from the donor molecule to the acceptor molecule can occur by a number of mechanisms, including, but not limited to, resonance dipole-induced dipole interaction, electron transfer, or charge transfer. Energy transfer only takes place over very short distances (typically less than 200 nm) and therefore the donor and acceptor molecules need so be in very close proximity.

As used herein, “Fluorescence Resonance Energy Transfer (FRET)” refers to non-radiative energy transfer between a donor and an acceptor molecule. Fluorescent resonance energy transfer (FRET) is an art-recognized process in which one fluorophore (the acceptor) can be promoted to an excited electronic state through quantum mechanical coupling with receipt of energy from an electronically excited second fluorophore (the donor). For FRET to occur efficiently, the absorption and emission spectra between the donor and acceptor generally have to overlap. Donor/acceptor pairs are characterized by their spectral overlap properties. Emission spectrum of the donor generally must overlap the acceptor absorption spectrum. Extent of overlap determines the efficiency of energy transfer. Extent of overlap also determines the optimal distance between the donor and acceptor molecule. Where the overlap of spectra is large, the transfer is efficient, so it can occur over long distances.

As used herein, “fluorescence” refers to emission of light that is caused by the absorption of radiation at one wavelength (excitation), followed by nearly immediate re-radiation (emission), usually at a different wavelength, that ceases almost at once when the incident radiation stops. At a molecular level, fluorescence occurs as certain compounds, known as fluorophores, are taken from a ground state to a higher state of excitation by light energy; as the molecules return to their ground state, they emit light, typically at a different wavelength (Lakowicz, J. R., “Principles of Fluorescence Spectroscopy,” (Plenum Press, NY, (1983)); Herman, B., “Resonance Energy Transfer Microscopy,” in: Fluorescence Microscopy of Living Cells in Culture, Part B, Methods in Cell Biology, vol. 30, (Taylor, D. L. & Wang, Y.-L., eds., (Academic Press, San Diego (1989), pp. 219-243).

As used herein, a “chromophore” refers to those groups that have favorable absorption characteristics, i.e., are capable of excitation upon irradiation by any of a variety of photonic sources. Chromophores can be fluorescing or non-fluorescing. Non-fluorescing chromophores typically do not emit energy in the form of photonic energy. Non-fluorescing chromophores can be characterized as having a low quantum yield, which is the ratio of emitted photonic energy to adsorbed photonic energy, typically less than 0.01.

As used herein, “fluorophore” refers to a fluorescent compound, such as a fluorescing chromophore. Fluorescence is a physical process in which light is emitted from the compound following absorption of radiation. Generally, the emitted light is of lower energy and longer wavelength than that absorbed. Fluorophores are molecules or moieties that fluoresce and/or are capable of generating a fluorescence signal. In particular, fluorophores are capable of absorbing energy, such as a photon, and re-emitting energy. Fluorophores typically emit photonic energy at medium to high quantum yields of 0.01 to 1. Sometimes the energy of the fluorophore is re-emitted as radiation usually of a longer wavelength than that which was absorbed by the fluorophore (i.e. fluorescence), and sometimes there is a time delay in the re-emission of the energy of the fluorophore (i.e. phosphorescence). Sometimes the energy of the fluorophore can be transferred to another molecule through a radiationless process (i.e. FRET).

As used herein, “excitation” refers to the absorption of radiation by a molecule resulting in an increase in the energy of the molecule and transition to a higher energy state.

As used herein, “emission” refers to the emission of a photon of energy by a molecule resulting in a decrease in the energy of the molecule and transition of a lower energy state.

As used herein, “energy transfer” refers to the transfer of energy among molecules such that the molecule that emits the energy transitions into a lower energy state while the second molecule that absorbs the energy emitted by the first molecule transitions into a higher energy state.

As used herein, “quenching group” or “quenching agent” refers to any fluorescence-modifying group of the invention that can attenuate at least partly the light emitted by a fluorescent group. As used herein, “quenching” refers to any process that causes a reduction in the quantum yield of a given fluorescence process. Hence, illumination of the fluorescent group in the presence of the quenching group leads to an emission signal that is less intense than expected, or even completely absent. Quenching typically occurs through energy transfer between the fluorescent group and the quenching group. The quenching group has the capacity to accept the transfer of energy of the donor molecule, but does not have significant emission. A quenching group includes an acceptor molecule that is configured to draw the energy potential away from an excited acceptor so that the acceptor does not emit.

As used herein, “EDANS” refers to the fluorophore 5-((2-aminoethyl)-amino)naphthalene-1-sulfonic acid.

As used herein, “DABCYL” refers to the acceptor chromophore 4-(4′-dimethylaminophenylazo)benzoic acid. As used herein, “DABSYL” refers to the acceptor chromophore 4-(4′-Dimethylamino-phenylazo)sulfonic acid.

As used herein, “energy transfer” refers to the process by which the fluorescence emission of a fluorescent group is altered by a fluorescence-modifying group. If the fluorescence-modifying group is a quenching group, then the fluorescence emission from the fluorescent group is attenuated or eliminated. Energy transfer can occur through fluorescence resonance energy transfer, or through direct energy transfer. The exact energy transfer mechanisms in these two cases are different. It is to be understood that any reference to energy transfer in the instant application encompasses all of these mechanistically-distinct phenomena

As used herein, “energy transfer pair” refers to any two molecules that participate in energy transfer. Typically, one of the molecules acts as a fluorescent group, and the other acts as a fluorescence-modifying group. In one embodiment, the energy transfer pair includes a fluorophore and a quenching group. In another embodiment, the energy transfer pair includes a photosensitizer and a quenching group. There is no limitation on the identity of the individual members of the energy transfer pair in this application. All that is required is that the spectroscopic properties of the energy transfer pair as a whole change in some measurable way if the distance between the individual members is altered by some critical amount.

As used herein, “fluorescence-modifying group” refers to a molecule that can alter in any way the fluorescence emission from a fluorescent group. A fluorescence-modifying group generally accomplishes this through an energy transfer mechanism. Depending on the identity of the fluorescence-modifying group, the fluorescence emission can undergo a number of alterations, including, but not limited to, attenuation, complete quenching, enhancement, a shift in wavelength, a shift in polarity, and a change in fluorescence lifetime. One example of a fluorescence-modifying group is a quenching group. If the fluorescence-modifying group is a quenching group, the quenching group usually does not radiate a substantial fraction of the absorbed light as light, and will generally dissipate it as heat.

As used herein, a “coordination cavity” or “coordination pocket” refers to the spatial arrangement of a chelated metal complex formed by the interaction of the ligands that bind to the metal. For example, in a porphyrin system, the coordination cavity is the “hole” in the macrocycle, the size of which is generally defined as the distance from the center to the mid-point of the four nitrogen atoms.

As used herein, “treatment” means any manner in which one or more of the symptoms of a disease or disorder are ameliorated or otherwise beneficially altered. Treatment also encompasses any pharmaceutical use of the conjugates herein, such as use for treating hyperproliferating tissue or neovascularization mediated diseases or disorders, or diseases or disorders in which hyperproliferating tissue or neovascularization is implicated.

As used herein, “amelioration of the symptoms” of a particular disorder by administration of a particular compound or pharmaceutical composition refers to any lessening, whether permanent or temporary, lasting or transient, that can be attributed to or associated with administration of the composition.

As used herein, “antibodies and antibody fragments” refers generally to immunoglobulins or fragments thereof that specifically bind to antigens to form immune complexes. The antibody may be whole immunoglobulin of any class, e.g. IgG, IgM, IgA, IgD, IgE, chimeric or hybrid antibodies with dual or multiple antigen or epitope specificities. It can be a polyclonal antibody, such as an affinity-purified antibody from a human or an appropriate animal, e.g., a primate, goat, rabbit, mouse or the like. Monoclonal antibodies are also suitable for use in the present invention, and are useful because of their high specificities. They are readily prepared by what are now considered conventional procedures of immunization of mammals with immunogenic antigen preparation, fusion of immune lymph or spleen cells with an immortal myeloma cell line, and isolation of specific hybridoma clones. More unconventional methods of preparing monoclonal antibodies are not excluded, such as interspecies fusions and genetic engineering manipulations of hypervariable regions, since it is primarily the antigen specificity of the antibodies that affects their utility. Newer techniques for production of monoclonals can also be used, e.g., human/monoclonals, interspecies monoclonals, chimeric (e.g., human/mouse) monoclonals, genetically engineered antibodies and the like.

As used herein, “tumor” denotes a neoplasm, and includes both benign and malignant tumors. This term particularly includes malignant tumors which can be either solid (such as a breast, liver, or prostate carcinoma) or non-solid (such as a leukemia). Tumors can also be further divided into subtypes, such as adenocarcinomas (e.g. of the breast, prostate or lung).

As used herein, “a target” denotes the object that is intended to be detected, diagnosed, impaired or destroyed by the methods provided herein, and includes target cells, target tissues, and target compositions. “Target tissues” and “target cells” as used herein are those tissues that are intended to be impaired or destroyed by this treatment method. Photosensitizing compounds bind to these target tissues or target cells; then when radiation appropriate to activate the photosensitizer is applied, these tissues or cells are impaired or destroyed. Target cells are cells in target tissue, and the target tissue includes, but is not limited to, vascular endothelial tissue, abnormal vascular walls of tumors, solid tumors such as (but not limited to) tumors of the head and neck, tumors of the eye, tumors of the gastrointestinal tract, tumors of the liver, tumors of the breast, tumors of the prostate, tumors of the lung, nonsolid tumors and malignant cells of the hematopoietic and lymphoid tissue, neovascular tissue, other lesions in the vascular system, bone marrow, and tissue or cells related to autoimmune disease. Also included among target cells are cells undergoing substantially more rapid division as compared to non target cells.

“Non-target tissues” as used herein are all the tissues of the subject which are not intended to be impaired or destroyed by the treatment method. These non-target tissues include but are not limited to healthy blood cells, and other normal tissue, not otherwise identified to be targeted.

“Target compositions” as used herein are those compositions that are intended to be impaired or destroyed by this treatment method, and may include one or more pathogenic agents, including but not limited to bacteria, viruses, fungi, protozoa, and toxins as well as cells and tissues infected or infiltrated therewith. The term “target compositions” also includes, but is not limited to, infectious organic particles such as prions, toxins, peptides, polymers, and other compounds that may be selectively and specifically identified as an organic target that is intended to be impaired or destroyed.

“Hyperproliferative tissue” as used herein means tissue that grows out of control and includes neoplastic tissue, tumors and unbridled vessel growth such as blood vessel growth found in age-related macular degeneration and often occurring after glaucoma surgeries.

“Hyperproliferative disorders” as used herein denotes those conditions sharing as an underlying pathology excessive cell proliferation caused by unregulated or abnormal cell growth, and include uncontrolled angiogenesis. Examples of such hyperproliferative disorders includes, but are not limited to, cancers or carcinomas, tumors, acute and membrano-proliferative glomerulonephritis, myelomas, psoriasis, atherosclerosis, psoriatic arthritis, rheumatoid arthritis, diabetic retinopathies, macular degeneration, corneal neovascularization, choroidal hemangioma, recurrence of pterygii, and scarring from excimer laser surgery and glaucoma filtering surgery.

As used herein, “amino acid” refers to natural or unnatural amino acids. The amino acids include but are not limited to 4-aminobutyric acid, 6-amino-hexanoic acid, alanine, asparagine, aspartic acid, arginine, 3-cyclohexyl-alanine, citruline, cysteine, 2,4-diaminobutyric acid, glutamine, glutamic acid, glycine, histidine, isoleucine, leucine, lysine, methionine, naphthylalanine, norleucine, ornithine, phenylalanine, 4-halogeno-phenylalanine, phenylglycine, proline, 3-(2-pyridyl)-alanine, serine, thienylalanine, threonine, tryptophan, tyrosine and valine.

A “therapeutically effective dose” or “therapeutically useful amount” as used herein is a dose sufficient to prevent advancement, or to cause regression of the disease, or which is capable of relieving symptoms caused by the disease.

A “pharmaceutical agent” or “drug” refers to a chemical compound or composition capable of inducing a desired therapeutic or prophylactic effect when properly administered to a subject.

Where relevant, chemical compounds include either of the (+) and (−) enantiomers, as well as the racemic mixture.

“Irradiating” and “irradiation” as used herein includes exposing a subject to all wavelengths of light. The irradiating wavelength is selected to include the wavelength(s) of light that excite the photosensitizer. In some embodiments, the radiation wavelength is selected to match the excitation wavelength of the photosensitizer and has low absorption by the non-target tissues of the subject, including blood proteins.

Irradiation is further defined herein by its coherence (laser) or non-coherence (non-laser), as well as intensity, duration, and timing with respect to dosing using the photosensitizing compound. The intensity or fluence rate must be sufficient for the light to reach the target tissue. The duration or total fluence dose must be sufficient to photoactivate enough photosensitizing compound to act on the target tissue. Timing with respect to dosing with the photosensitizing compound is important, because 1) the administered photosensitizing compound requires some time to home in on target tissue and 2) the blood level of many photosensitizing compounds decreases with time. The radiation energy is provided by an energy source, such as a laser or cold cathode light source, that is external to the subject, or that is implanted in the subject, or that is introduced into a subject, such as by a catheter, optical fiber or by ingesting the light source in capsule or pill form (e.g., as disclosed in. U.S. Pat. No. 6,273,904 (2001)).

While one embodiment of the present invention is drawn to the use of light energy for administering PDT to destroy tumors, other forms of energy are within the scope of this invention, as will be understood by those of ordinary skill in the art. Such forms of energy include, but are not limited to: thermal, sonic, ultrasonic, chemical, electromagnetic radiation, mechanical, and electrical. For example, sonodynamically induced or activated agents include, but are not limited to: gallium porphyrin complex (see Yumita et al., Cancer Letters 112: 79-86 (1997)), other porphyrin complexes, such as protoporphyrin and hematoporphyrin (see Umemura et al., Ultrasonics Sonochemistry 3: S187-S191 (1996)); other cancer drugs, such as daunorubicin and adriamycin, used in the presence of ultrasound therapy (see Yumita et al, Japan J. Hyperthermic Oncology 3(2):175-182 (1987)).

As used herein, “destroy” or “destruction” means to kill the desired target tissue or target composition, including infecting agents. “Impair” or “impairment” means to change the target tissue or target composition in such a way as to interfere with its function or reduce its growth. For example, in North et al., it is observed that after virus infected T cells treated with benzoporphyrin derivatives were exposed to light, holes developed in the T cell membrane and increased in size until the membrane completely decomposed (Blood Cells 18:129 40 (1992)). The target tissue or target composition is understood to be impaired or destroyed even if the target tissue or target composition is ultimately disposed of by macrophages.

The present invention provides a method for providing a medical therapy to an animal, and the term “animal” includes, but is not limited to, humans and other mammals.

The term “coupling agent” as used herein, refers to a reagent capable of coupling a photosensitizer to a targeting agent. The term “linking agent” or “linking component” as used herein, refers to a reagent capable of linking a photosensitizer to a targeting agent. In some embodiments, the “linking component” may also serve as the targeting moiety.

As used herein, “targeting agent” or “targeting moiety” refers to a compound that homes in on or preferentially associates or binds to a particular tissue, receptor, infecting agent or other area of the body of the subject to be treated, such as a target tissue or target composition. Examples of a targeting agent include but are not limited to an oligonucleotide, an antigen, an antibody, a ligand, a receptor, one member of a specific binding pair, a polyamide including a peptide having affinity for a biological receptor, an oligosaccharide, a polysaccharide, a low density lipoprotein (LDL) or the APO-protein of LDL, a steroid or steroid derivative, a hormone such as estradiol or histamine, a hormone-mimic such as morphine, or other compound having binding specificity for a target.

As used herein, “specific binding pair” and “ligand-receptor binding pair” refers to two different molecules, where one of the molecules has an area on the surface or in a cavity which specifically attracts or binds to a particular spatial or polar organization of the other molecule, causing both molecules to have an affinity for each other. The members of the specific binding pair are referred to as ligand and receptor (anti-ligand). The terms ligand and receptor are intended to encompass the entire ligand or receptor or portions thereof sufficient for binding to occur between the ligand and the receptor. Examples of ligand-receptor binding pairs include, but are not limited to, hormones and hormone receptors, for example epidermal growth factor and epidermal growth factor receptor, tumor necrosis factor-□ and tumor necrosis factor-receptor, and interferon and interferon receptor; avidin and biotin or anti-biotin; antibody and antigen pairs; enzymes and substrates, drug and drug receptor; cell-surface antigen and lectin; two complementary nucleic acid strands; nucleic acid strands and complementary oligonucleotides; interleukin and interleukin receptor, and stimulating factors and their receptors, such as granulocyte-macrophage colony stimulating factor (GMCSF) and GMCSF receptor and macrophage colony stimulating factor (MCSF) and MCSF receptor.

As used herein, a “receptor” refers to a molecule that has an affinity for a given ligand. Receptors can be naturally-occurring or synthetic molecules. Receptors can also be referred to in the art as anti-ligands. As used herein, the receptor and anti-ligand are interchangeable. Receptors can be used in their unaltered state or as aggregates with other species. Receptors can be attached, covalently or noncovalently, or in physical contact with, to a binding member, either directly or indirectly via a specific binding substance or linker. Examples of receptors, include, but are not limited to: antibodies, cell membrane receptors surface receptors and internalizing receptors, monoclonal antibodies and antisera reactive with specific antigenic determinants, such as on viruses, cells, or other materials, drugs, polynucleotides, nucleic acids, peptides, cofactors, lectins, sugars, polysaccharides, cells, cellular membranes, and organelles.

As used herein, “specific binding” or “selective binding” means that the binding of a targeting agent and its target is greater than for a non-target, such as another receptor. A statement that a particular compound is targeted to a target cell or target tissue means that its affinity for such cell or tissue in a host or in vitro or in vivo is greater than for other cells and tissues in the host or under the in vitro conditions.

As used herein, “sample” refers to anything that contains a target for which a target assay is desired. The sample can be a biological sample, such as a biological fluid or a biological tissue. Examples of biological fluids include urine, blood, plasma, serum, saliva, semen, stool, sputum, cerebral spinal fluid, tears, mucus, sperm, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, “pharmaceutically acceptable derivatives” of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The conjugates may be administered to animals or humans without substantial toxic effects and either are pharmaceutically active or are prodrugs.

Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl.

As used herein, “treatment” means any manner in which one or more of the symptoms of a disease or disorder are ameliorated or otherwise beneficially altered. Treatment also encompasses any pharmaceutical use of the conjugates herein, such as use for treating hyperproliferating tissue or neovascularization mediated diseases or disorders, or diseases or disorders in which hyperproliferating tissue or neovascularization is implicated.

As used herein, an “effective amount” of a compound for treating a particular disease is an amount that is sufficient to ameliorate, or in some manner reduce the symptoms associated with the disease. Such amount can be administered as a single dosage or can be administered according to a regimen, whereby it is effective. The amount can cure the disease but, typically, is administered in order to ameliorate the symptoms of the disease. Repeated administration can be required to achieve the desired amelioration of symptoms.

As used herein, “combination” refers to any association between 2 or more items.

As used herein, a “kit” is a packaged combination, where elements of a combination are contained within a package, optionally including instructions and reagents.

As used herein, a “composition” refers to any mixture. It can be a solution, a suspension, liquid, powder, a paste, aqueous, non-aqueous or any combination thereof.

As used herein, “fluid” refers to any composition that can flow. Fluids thus encompass compositions that are in the form of semi-solids, pastes, solutions, aqueous mixtures, gels, lotions, creams and other such compositions.

The conjugates, kits, articles of manufacture and methods discussed in the following sections are generally representative of the disclosed conjugates and the methods in which such conjugates can be used. The following discussion is intended as illustrative of selected aspects and embodiments of the present invention and it should not be interpreted as limiting the scope of the present disclosure.

B. Conjugates

Conjugates for enhancing the action of fluorescence detection or photodynamic therapy for the purpose of detecting or destroying tumors, hyperproliferative tissue, or other undesired biological structures are disclosed herein. In order to minimize unwanted activity of a donor molecule, such as a fluorophore or a targeted photosensitizer for PDT, and improve selectivity of the donor molecule when used for diagnostic purposes, the donor molecule is made part of a larger molecule or conjugate into which at least two other parts and the necessary linking components are incorporated. In one embodiment, the first of these components is a targeting moiety (TM), which can be an antibody or any other ligand or binding agent possessing the desired binding affinity and specificity for the target cell or structure. The second component incorporated is an acceptor molecule, such as a quenching agent (QA) built into the conjugate in such a way that the quenching agent is in a position from which it can effectively quench (or dissipate the energy of, usually in the form of thermal energy transferred to the medium) the excited state of the sensitizer when it is not bound to its intended target.

1. Energy Transfer Pair

a. Donor Molecule

i. Fluorophores

In one embodiment, the donor molecule is a fluorophore. A fluorophore is a fluorescing chromophore, or a molecule that emits light at a given wavelength when stimulated by absorption of light of a different wavelength. Any fluorophore known in the art is useful in the disclosed conjugates. Exemplary compounds include, but are not limited to, cyanine, indocarbocyanine, tetramethyl rhodamine, indodicarbocyanine, carbocyanine, calcein, FITC, rhodamine 110, 5-carboxyfluorescein, fluorescein succinimidyl esters, 2′,7′-difluorofluorescein, carboxyfluorescein succinimidyl ester, 6-carboxy-4′,5′-dichloro-2′,7′-dimethoxyfluorescein ester, 6-carboxy-2′,4,7,7′-tetrachlorofluorescein succinimidyl ester, 6-carboxy-2′,4,4′,5′,7,7′-hexachloro-fluorescein ester, rhodamine green, phycoerythrin, rhodamine phalloidin, rhodamine B, rhodamine red-X, X-rhodamine, sulforhodamine 101, Pyronin Y, TAMRA, ROX, R-phycocyanin, C-Phycocyanin, and thiadicarbocyanine. When the conjugate is for in vivo use, the fluorophores of the composition are generally selected to absorb light in the near infrared spectrum (600-1000 run) to maximize tissue penetration by minimizing absorption by physiologically abundant absorbers such as hemoglobin (<550 nm) or water (>1200 nm). A variety of such fluorophores are known in the art, including, but not limited to allophycocyanin; indodicarbocyanine; indotricarbocyanine; thiadicarocynine; fluorescein, sulforhodamine; ROX; sulforhodamine; nile red; R-phycocyanin; C-phycocyanin; and thiadicarbocyanine. Many other fluorophores are commercially available from, for example, Frontier Scientific (Logan, Utah.); the SIGMA Chemical Company (Saint Louis, Mo.), Molecular Probes (Eugene, Oreg.), R&D Systems (Minneapolis, Minn.), Pharmacia LKB Biotechnology (Piscataway, N.J.), CLONTECH Laboratories, Inc. (Palo Alto, Calif), Aldrich Chemical Company Milwaukee, Wis.), GIBCO BRL Life Technologies, Inc. (Gaithersburg, Md.), Fluka Chemica-Biochemika Analytika (Fluka Chemie AG, Buchs, Switzerland), and Applied Biosystems (Foster City, Calif.), as well as many other commercial sources known to one of skill.

ii. Photosensitizing Agents

In another embodiment, the donor molecule is a photosensitizing agent. A photosensitizing agent is a chemical compound that upon exposure to photoactivating light is activated, converting the photosensitizing agent into a cytotoxic form, whereby target cells are killed or their proliferative potential diminished. The photosensitizing agent of the conjugates disclosed herein can be any of the variety of synthetic and naturally occurring photosensitizing agents known in the art, including pyrrole based photosensitizing agents such as porphyrins and porphyrin derivatives, e.g. chlorins, bacteriochlorins, isobacteriochlorins, phthalocyanine and naphthalocyanines and other tetra- and poly-macrocyclic compounds, and related compounds (e.g. pyropheo-phorbides, sapphyrins and texaphyrins) and metal complexes (such as, but not limited by, tin, aluminum, zinc, lutetium). Tetrahydrochlorins, purpurins, porphycenes, and phenothiaziniums are also within the scope of the disclosure. Generally, any polypyrrolic macrocyclic photosensitive compound that is hydrophobic can be used.

Examples of these and other photosensitizing agents include, but are not limited to, angelicins, chalcogenapyrillium dyes, chlorins, chlorophylls, coumarins, cyanines, ceratin daunomycin; daunomycinone; 5-iminodauno-mycin; doxycycline; furosemide; gilvocarcin M; gilvocarcin V; hydroxy-chloroquine sulfate; lumidoxycycline; mefloquine hydrochloride; mequitazine; merbromin (mercurochrome); primaquine diphosphate; quinacrine dihydrochloride; quinine sulfate; and tetracycline hydrochloride, certain flavins and related compounds such as alloxazine; flavin mononucleotide; 3-hydroxyflavone; limichrome; limiflavin; 6-methylalloxazine; 7-methylalloxazine; 8-methylalloxazine; 9-methylalloxazine; 1-methyl limichrome; methyl-2-methoxybenzoate; 5-nitrosalicyclic acid; proflavine; and riboflavin, metallo-porphyrins, metallophthalocyanines, methylene blue derivatives, naphthalmides, naphthalocyanines, pheophorbides, pheophytins, photosensitizer dimers and conjugates, phthalocyanines, porphycenes, porphyrins, psoralens, purpurins, quinones, retinoids, rhodamines, thiophenes, verdins, vitamins and xanthene dyes (Redmond and Gamlin, Photochem. Photobiol., 70(4):391-475 (1999)).

(a) Exemplary Metalloporphyrins

Exemplary metalloporphyrins include cobalt meso-tetra-(4-N-methylpyridyl)-porphine; cobalt (II) meso(4-sulfonatophenyl)-porphine; copper hematoporphyrin; copper meso-tetra-(4-N-methylpyridyl)-porphine; copper (II) meso(4-sulfonatophenyl)-porphine; Europium (III) dimethyltexaphyrin dihydroxide; gallium tetraphenylporphyrin; iron meso-tetra(4-N-methylpyridyl)-porphine; lutetium (III) tetra(N-methyl-3-pyridyl)-porphyrin chloride; magnesium (II) meso-diphenyl tetrabenzoporphyrin; magnesium tetrabenzoporphyrin; magnesium tetraphenylporphyrin; magnesium (II) meso(4-sulfonatophenyl)-porphine; magnesium (II) texaphyrin hydroxide metalloporphyrin; magnesium meso-tetra-(4-N-methylpyridyl)-porphine; manganese meso-tetra-(4-N-methyl-pyridyl)-porphine; nickel meso-tetra(4-N-methylpyridyl)-porphine; nickel (II) meso-tetra(4-sulfonatophenyl)-porphine; palladium (II) meso-tetra-(4-N-methylpyridyl)-porphine; palladium meso-tetra-(4-N-methylpyridyl)-porphine; palladium tetraphenylporphyrin; palladium (II) meso(4-sulfonatophenyl)-porphine; platinum (II) meso(4-sulfonatophenyl)-porphine; samarium (II) dimethyltexaphyrin dihydroxide; silver (II) meso(4-sulfonatophenyl)-porphine; tin (IV) protoporphyrin; tin meso-tetra-(4-N-methylpyridyl)-porphine; tin meso-tetra(4-sulfonatophenyl)-porphine; tin (IV) tetrakis(4-sulfonatophenyl) porphyrin dichloride; cadmium (II) chlorotexaphyrin nitrate; cadmium (II) meso-diphenyl tetrabenzoporphyrin; cadmium meso-tetra-(4-N-methylpyridyl)-porphine; cadmium (II) texaphyrin; cadmium (II) texaphyrin nitrate; zinc (II) 15-aza-3,7,12,18-tetramethyl-porphyrinato-13,17-diyl-dipropionic acid-dimethylester; zinc (II) chlorotexaphyrin chloride; zinc coproporphyrin III; zinc (II) 2,11,20,30-tetra-(1,1-dimethyl-ethyl)tetranaphtho(2,3-b:2′,3′-g:2″3″-1:2′″3′″-q)porphyrazine; zinc (II) 2-(3)-pyridyloxy)benzo[b]-10,19,28-tri(1,1-dimethylethyl)trinaphtho[2′,3′-g:2″3″1::2′″,3′″-q]porphyrazine; zinc (II) 2,18-bis-(3-pyridyloxy)dibenzo[b,1]-10,26-di(1,1-dimethyl-ethyl)dinaphtho[2′,3′-g:2′″,3′″-q]porphyrazine; zinc (II) 2,9-bis-(3-pyridyloxy)dibenzo[b,g]-17,26-di(1,1-dimethyl-ethyl)dinaphtho[2″,3″-1:2′″,3′″-q]porphyrazine; zinc (II) 2,9,16-tris-(3-pyridyloxy)tribenzo[b,g,1]-24=(1,1-dimethyl-ethyl)naphtho[2′″,3′″-q]porphyrazine; zinc (II) 2,3-bis-(3-pyridyloxy)benzo[b]-10,19,28-tri(1,1-dimethyl-ethyl)trinaphtho[2′,3′-g:2″,3″1:2′″,3′″-q]porphyrazine; zinc (II) 2,3,18,19-tetrakis-(3-pyridyloxy)dibenzo[b,1]-10,26-di(1,1-dimethyl-ethyl)trinaphtho[2′,3′-g:2′″,3′″-q]porphyrazine; zinc (II) 2,3,9,10-tetrakis-(3-pyridyloxy)dibenzo[b,g]-17,26-di(1,1-dimethyl-ethyl)dinaphtho[2″,3″-1:2′″,3′″-q]porphyrazine; zinc (II) 2,3,9,10,16,17-hexakis-(3-pyridyloxy)tribenzo[b,g,1]-24(1,1-dimethyl-ethyl)naphtho[2′″,3′″-q]porphyrazine; zinc (II) 2-(3-N-methyl)pyridyloxy)benzo[b]-10,19,28-tri(1,1-dimethyl-ethyl)trinaphtho[2′,3′-g:2″,3″1:2′″,3′″-q]porphyrazine monoiodide; zinc (II) 2,18-bis-(3-(N-methyl)pyridyloxy)dibenzo[b,1]-10,26-di(1,1-dimethylethyl)dinaphtho[2′,3′-g:2′″,3′″-q]porphyrazine diiodide; zinc (II) 2,9-bis-(3-(N-methyl)pyridyloxy)dibenzo[b,g]-17,26-di(1,1-dimethylethyl)dinaphtho[2″,3″-1:2′″,3′″-q]porphyrazine diiodide; zinc (II) 2,9,16-tris-(3-(N-methyl-pyridyloxy)tribenzo[b,g,1]-24-(1,1-dimethylethyl)naphtho[2′″,3′″-q]porphyrazine triiodide; zinc (II) 2,3-bis-(3-(N-methyl)pyridyloxy)benzo[b]-10,19,28-tri(1,1-dimethylethyl)trinaphtho[2′,3′-g:2″,3″-1:2′″,3′″-q]porphyrazine diiodide; zinc (II) 2,3,18,19-tetrakis-(3-(N-methyl)pyridyloxy)dibenzo[b,1]-10,26-di(1,1-dimethyl)dinaphtho[2′,3′-g:2′″,3′″-q]porphyrazine tetraiodide; zinc (II) 2,3,9,10-tetrakis-(3-(N-methyl)pyridyloxy)dibenzo[g,g]-17,26-di(1,1-dimethylethyl)dinaphtho[2″,341 -1:2′″,3′″-q]porphyrazine tetraiodide; zinc (II) 2,3,9,10,16,17-hexakis-(3-(N-methyl)pyridyloxy)tribenzo[b,g,1]-24-(1,1-dimethylethyl)naphtho[2′″,3′″-q]porphyrazine hexaiodide; zinc (II) meso-diphenyl tetrabenzoporphyrin; zinc (II) meso-triphenyl tetrabenzoporphyrin; zinc (II) meso-tetrakis(2,6-dichloro-3-sulfonatophenyl) porphyrin; zinc (II) meso-tetra-(4-N-methylpyridyl)-porphine; zinc (II) 5,10,15,20-meso-tetra(4-octyl-phenylpropynyl)-porphine; zinc porphyrin c; zinc protoporphyrin; zinc protoporphyrin IX; zinc (II) meso-triphenyl-tetrabenzoporphyrin; zinc tetrabenzoporphyrin; zinc (II) tetrabenzoporphyrin; zinc tetranaphthaloporphyrin; zinc tetraphenylporphyrin; zinc (II) 5,10,15,20-tetraphenylporphyrin; zinc (II) meso (4-sulfonatophenyl)-porphine; and zinc (II) texaphyrin chloride.

(b) Exemplary Pheophorbides

Exemplary pheophorbides include pheophorbide a; methyl 13-1-deoxy-20-formyl-7,8-vic-dihydro-bacterio-meso-pheophorbide a; methyl-2-(1-dodecyloxyethyl)-2-devinyl-pyropheophorbide a; methyl-2-(1-heptyl-oxyethyl)-2-devinyl-pyropheophorbide a; methyl-2-(1-hexyl-oxyethyl)-2-devinyl-pyropheophorbide a; methyl-2-(1-methoxy-ethyl)-2-devinyl-pyropheophorbide a; methyl-2-(1-pentyl-oxyethyl)-2-devinyl-pyropheophorbide a; magnesium methyl bacteriopheophorbide d; methyl-bacteriopheophorbide d; and pheophorbide.

(c) Exemplary Porphyrins

Exemplary porphyrins include 5-azaprotoporphyrin dimethylester; bis-porphyrin; coproporphyrin III; coproporphyrin III tetramethylester; deuteroporphyrin; deuteroporphyrin IX dimethylester; diformyldeutero-porphyrin IX dimethylester; dodecaphenylporphyrin; hematoporphyrin; hematoporphyrin; hematoporphyrin; hematoporphyrin; hematoporphyrin; hematoporphyrin; hematoporphyrin; hematoporphyrin; hematoporphyrin IX; hematoporphyrin monomer; hematoporphyrin dimer; hematoporphyrin derivative; hematoporphyrin derivative; hematoporphyrin derivative; hematoporphyrin derivative A; hematoporphyrin IX dihydrochloride; hematoporphyrin dihydrochloride; hematoporphyrin IX dimethylester; haematoporphyrin IX dimethylester; mesoporphyrin dimethylester; mesoporphyrin IX dimethylester; monoformyl-monovinyl-deuteroporphyrin IX dimethylester; monohydroxyethylvinyl deuteroporphyrin; 5,10,15,20-tetra(o-hydroxyphenyl)porphyrin; 5,10,15,20-tetra(m-hydroxyphenyl)porphyrin; 5,10,15,20-tetrakis-(m-hydroxyphenyl)-porphyrin; 5,10,15,20-tetra(p-hydroxyphenyl) porphyrin; 5,10,15,20-tetrakis (3-methoxyphenyl)-porphyrin; 5,10,15,20-tetrakis (3,4-dimethoxyphenyl)porphyrin; 5,10,15,20-tetrakis (3,5-dimethoxyphenyl)porphyrin; 5,10,15,20-tetrakis (3,4,5-trimethoxyphenyl)porphyrin; 2,3,7,8,12,13,17,18-octaethyl-5,10,15,20-tetraphenylporphyrin; Photofrin®; Photofrin II; porphyrin c; protoporphyrin; protoporphyrin IX; protoporphyrin dimethylester; protoporphyrin IX dimethylester, protoporphyrin propylaminoethylformamide iodide; protoporphyrin N,N-dimethylaminopropyl-formamide; protoporphyrin propylaminopropylformamide iodide; protoporphyrin butylformamide; protoporphyrin N,N-dimethylamino-formamide; protoporphyrin formamide; sapphyrin 13,12,13,22-tetraethyl-2,7,18,23 tetramethyl sapphyrin-8,17-dipropanol; sapphyrin 23,12,13,22-tetraethyl-2,7,18,23 tetramethyl sapphyrin-8-monoglycoside; sapphyrin 3; meso-tetra-(4-N-carboxyphenyl)-porphine; tetra-(3-methoxyphenyl)-porphine; tetra-(3-methoxy-2,4-difluorophenyl)-porphine; 5,10,15,20-tetrakis(4-N-methylpyridyl)porphine; meso-tetra-(4-N-methylpyridyl)-porphine tetrachloride; meso-tetra(4-N-methylpyridyl)-porphine; meso-tetra-(3-N-methylpyridyl)-porphine; meso-tetra-(2-N-methylpyridyl)-porphine; tetra(4-N,N,N-trimethylanilinium)porphine; meso-tetra-(4-N,N,N″-trimethylamino-phenyl)porphine tetrachloride; tetranaphthaloporphyrin; 5,10,15,20-tetraphenylporphyrin; tetraphenylporphyrin; meso-tetra-(4-N-sulfonatophenyl)-porphine; tetraphenylporphine tetrasulfonate; meso-tetra(4-sulfonatophenyl)-porphine; tetra(4-sulfonatophenyl)porphine; tetraphenylporphyrin sulfonate; meso-tetra(4-sulfonatophenyl)porphine; tetrakis (4-sulfonatophenyl)porphyrin; meso-tetra(4-sulfonatophenyl)porphine; meso(4-sulfonatophenyl)porphine; meso-tetra(4-sulfonatophenyl)porphine; tetrakis(4-sulfonatophenyl)porphyrin; meso-tetra(4-N-trimethylanilinium)-porphine; uroporphyrin; uroporphyrin I; uroporphyrin IX; and uroporphyrin I.

The photosensitizing agents for use in the conjugates disclosed herein include porphyrin derivatives obtained by reacting a porphyrin nucleus with an alkyne in a Diels-Alder type reaction to obtain a monohydrobenzo-porphyrin, such as those described in detail by Levy et al. in U.S. Pat. No. 5,171,749, which is hereby incorporated in its entirety by reference. The absorption spectrum of the photosensitizing agent is typically between 400 nm and 1200 nm, and in some embodiments between 500-900 nm or between 600-900 nm.

(d) Exemplary Psoralens

Exemplary psoralens include psoralen; 5-methoxypsoralen; 8-methoxy-psoralen; 5,8-dimethoxypsoralen; 3-carbethoxypsoralen; 3-carbethoxy-pseudopsoralen; 8-hydroxypsoralen; pseudopsoralen; 4,5′,8-trimethyl-psoralen; allopsoralen; 3-aceto-allopsoralen; 4,7-dimethyl-allopsoralen; 4,7,4′-trimethyl-allopsoralen; 4,7,5′-trimethyl-allopsoralen; isopseudopsoralen; 3-acetoisopseudopsoralen; 4,5′-dimethyl-isopseudo-psoralen; 5′,7-dimethyl-isopseudopsoralen; pseudoisopsoralen; 3-aceto-seudoisopsoralen; 3/4′,5′-trimethyl-aza-psoralen; 4,4′,8-trimethyl-5′-amino-methylpsoralen; 4,4′,8-trimethyl-phthalamyl-psoralen; 4,5′,8-trimethyl-4′-aminomethyl psoralen; 4,5′,8-trimethyl-bromopsoralen; 5-nitro-8-methoxy-psoralen; 5′-acetyl4,8-dimethyl-psoralen; 5′-aceto-8-methyl-psoralen; and 5′-aceto-4,8-dimethyl-psoralen. Exemplary purpurins include octaethylpurpurin; octaethylpurpurin zinc; oxidized octaethylpurpurin; reduced octaethylpurpurin; reduced octaethylpurpurin tin; purpurin 18; purpurin-18; purpurin-18-methyl ester; purpurin; tin ethyl etiopurpurin I; Zn(II) aetio-purpurin ethyl ester; and zinc etiopurpurin.

(e) Exemplary Quinones

Exemplary quinones include 1-amino-4,5-dimethoxy anthraquinone; 1,5-diamino-4,8-dimethoxy anthraquinone; 1,8-diamino-4,5-dimethoxy anthraquinone; 2,5-diamino-1,8-dihydroxy anthraquinone; 2,7-diamino-1,8-dihydroxy anthraquinone; 4,5-diamino-1,8-dihydroxy anthraquinone; mono-methylated 4,5- or 2,7-diamino-1,8-dihydroxy anthraquinone; anthralin (keto form); anthralin; anthralin anion; 1,8-dihydroxy anthraquinone; 1,8-dihydroxy anthraquinone (Chrysazin); 1,2-dihydroxy anthraquinone; 1,2-dihydroxy anthraquinone (Alizarin); 1,4-dihydroxy anthraquinone (Quinizarin); 2,6-dihydroxy anthraquinone; 2,6-dihydroxy anthraquinone (Anthraflavin); 1-hydroxy anthraquinone (Erythroxy-anthraquinone); 2-hydroxy-anthraquinone; 1,2,5,8-tetra-hydroxy anthraquinone (Quinalizarin); 3-methyl-1,6,8-trihydroxy anthraquinone (Emodin); anthraquinone; anthraquinone-2-sulfonic acid; benzoquinone; tetramethyl benzoquinone; hydroquinone; chlorohydroquinone; resorcinol; and 4-chlororesorcinol.

(f) Exemplary Retinoids

Exemplary retinoids include all-trans retinal; C₁₇ aldehyde; C₂₂ aldehyde; 11-cis retinal; 13-cis retinal; retinal; and retinal palmitate.

(g) Exemplary Rhodamines

Exemplary rhodamines include 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester.

(h) Examples of Other Photosensitizers

Other non-limiting examples of photosensitizing agents that may be useful in the conjugates are bacteriochlorophyll-A derivatives, described in U.S. Pat. Nos. 5,171,741 and 5,173,504; photosensitizing Diels-Alder porphyrin derivatives, described in U.S. Pat. No. 5,308,608; porphyrin-like compounds, described in U.S. Pat. Nos. 5,405,957, 5,512,675, and 5,726,304; imines of porphyrin and porphyrin derivatives, as described in U.S. Pat. Nos. 5,424,305 and 5,744,598; alkyl ether analogs of benzoporphyrin derivatives, as described in U.S. Pat No. 5,498,710; purpurin-18, bacteriopurpurin-18 and related compounds, as described in U.S. Pat. No. 5,591,847; meso-substituted chorins, isobacteriochlorins and bacteriochlorins, as described in U.S. Pat. No. 5,648,485; meso-substituted tetramacrocyclic compounds, as described in U.S. Pat. No. 5,703,230; carbodiimide analogs of chlorins and bacteriochlorins, as described in U.S. Pat. No. 5,770,730; meso-substituted chlorins, isobacteriochlorins and bacteriochlorins, as described in U.S. Pat. No. 5,831,088; polypyrrolic macrocycles from meso-substituted tripyrrane compounds, described in U.S. Pat. Nos. 5,703,230, 5,883,246, and 5,919,923; isoimides of chlorins and bacteriochlorins, described in U.S. Pat. No. 5,864,035; alkyl ether analogs of chlorins having an N-substituted imide ring, as described in U.S. Pat. No. 5,952,366; ethylene glycol esters, described in U.S. Pat. No. 5,929,105; carotene analogs of porphyrins, chlorins and bacteriochlorins, as described in U.S. Pat. No. 6,103,751; fatty acid ester derivatives of porphyrin, chlorin, or bacteriochlorin, as described in U.S. Pat. No. 6,245,811; indium photosensitizers, as described in U.S. Pat. No. 6,444,194; porphyrins, chlorins, bacteriochlorins, and related tetrapyrrolic compounds described in U.S. Pat. No. 6,534,04; 1,3-propane diol ester and ether derivatives of porphyrins, chlorins and bacteriochlorins, as described in U.S. Pat. No. 6,555,700; trans beta substituted chlorins, as described in U.S. Pat. No. 6,559,374; and palladium-substituted bacteriochlorophyl derivatives, as described in U.S. Pat. No. 6,569,846; and the photosensitizer entities disclosed in Wilson et al. (Curr. Micro. 25:77-81, 1992) and in Okamoto et al. (Lasers in Surg. Med. 12:450-485, 1992). Generally any hydrophobic or hydrophilic photosensitizing agent, which absorbs in the ultra-violet, visible and infra-red spectroscopic ranges, would be useful in the disclosed conjugates.

b. Acceptor Molecule

The acceptor molecule of the disclosed conjugate is a chemical or biological compound that is capable of receiving or accepting energy from another molecule. In one embodiment, the conjugate disclosed herein includes as an acceptor molecule a quenching agent. Any fluorescence-modifying group that can attenuate at least partly the light emitted by a fluorophore or prevent activation of a photosensitizing agent can be used as a quenching agent in the disclosed conjugates. This attenuation typically occurs through energy transfer between the donor molecule, such as a fluorophore or photosensitizing agent, and the acceptor molecule, such as a quenching agent.

Fluorescence quenching commonly takes place by a number of mechanisms, including direct and indirect energy transfer. In all cases, when donor molecule of the disclosed conjugate, which includes a fluorophore or photosensitizing agent, is excited by input of energy, typically by irradiation with a specific wavelength of light, energy is transferred from donor molecule, such as a fluorophore or a photosensitizing agent, to the acceptor molecule, such as a quenching agent, rather than being dissipated by fluorescence or conversion of the photosensitizing agent into an active state. The quenching agent, as an acceptor molecule, has the capacity to accept the transfer of energy, for example by dipole coupling, but does not have significant emission.

The quenching agent is therefore any chemical that can transfer or dissipate the energy of the excited state of the donor molecule, such as a fluorophore or a photosensitizing agent of the conjugate, when the conjugate is not bound to its intended target. Quenching agents include, but are not limited to, acceptor chromophores that do not demonstrate significant emission, and aromatic compounds capable of accepting transferred energy, such as nitrosated aromatic compounds, including nitrophenyl, nitrobenzyloxycarbonyl, nitrobenzoyl.

Exemplary Quenching Agents

Exemplary quenching agents include 4-(4′-dimethylamino-phenylazo)benzoic acid (DABCYL); dabcyl succinimidyl ester; 4-(4′-dimethylamino-phenylazo)sulfonic (DABSYL); dabsyl succinimidyl ester; tetramethyl-rhodamaine (TAMRA); 4-[(4-nitrophenyl)diazinyl]-phenylamine and 4-[4-nitrophenyl)diazinyl]-naphthylamine; dabcylnitro-thiazole; 6-(N-[7-nitrobenz-2-oxa-1,3-diazol-4-yl]amino) hexanoic acid; 6-carboxy-X-rhodamine (ROX);QSY-7; 2-[4-(4-nitrophenylazo)-N-ethylphenyl-amino]ethanol (Disperse Red 1); 2-[4-(2-chloro-4-nitrophenyl-azo)-N-ethylphenylamino]-ethanol (Disperse Red 13); tetrarhodamine isothiocyanate (TRITC); allophycocyanin; β-carotene; diarylrhodamine derivatives, such as the QSY 7, QSY 9, and QSY 21 dyes; QSY 35 acetic acid succinimidyl ester; QSY 35 iodoacetamide and aliphatic methylamine; napthalate; Reactive Red 4; and Malachite Green.

There is a great deal of practical guidance available in the literature for selecting appropriate donor-acceptor pairs for use in the disclosed conjugates. For example, see Pesce et al., “Fluorescence Spectroscopy” (Marcel Dekker, New York, 1971); White et al.,

“Fluorescence Analysis: A Practical Approach” (Marcel Dekker, New York, 1970). The literature also includes references providing exhaustive lists of fluorescent and chromogenic molecules and their relevant optical properties, for choosing reporter-quencher (donor-acceptor) pairs (see, for example, Berlman, “Eandbook of Fluorescence Spectra of Aromatic Molecules,” 2nd Edition (Academic Press, New York, 1971); Griffiths, “Color and Constitution of Organic Molecules, “(Academic Press, New York, 1976); Bishop, “Indicators” (Pergamon Press, Oxford, 1972); and Haugland, “Handbook of Fluorescent Probes and Research Chemicals,” (Molecular Probes, Eugene, 1992).

c. Selection of the Energy Transfer Pair

The ability of the donor molecule to transfer energy to an acceptor molecule depends on a number of factors. These include, but are not limited to, the energy transfer efficiency, the spectral overlap between the acceptor and the donor molecule, dipole, fluorescence quantum yield of the donor, the extinction coefficient of acceptor, and the fluorescence emission intensity of donor. Because these factors are dependent on the environment, the actual value observed in a specific experimental situation is somewhat variable.

i. Fluorescence Resonance Energy Transfer (FRET)

FRET refers to non-radiative energy transfer between chemical and/or biological luminescent molecules (Heim et al., Curr. Biol. 6:178-182 (1996); Mitra et al. Gene 173:13-17 (1996); Selvin et al., Meth. Enzymol. 246:300-345 (1995); Matyus, J. Photochem. Photobiol. B: Biol. 12: 323-337 (1992); Wu et al., Anal. Biochem. 218:1-13 (1994)). The efficiency of FRET is dependent on the inverse sixth power of the intermolecular separation making it useful over distances comparable with the dimensions of biological macromolecules (Stryer and Haugland, Proc Natl Acad Sci USA 58: 719-726 (1967)). Thus, The sensitivity of FRET to molecular proximity has been described (dos Remedios et al., J Struct Biol 115: 175-185 (1995); Selvin Methods Enzymol 246: 300-334 (1995); Boyde et al., Scanning 17: 72-85 (1995); Wu et al., Anal Biochem 218: 1-13 (1994); Van der Meer et al., “Resonance Energy Transfer Theory and Data,” pp. 133-168 (1994); Kawski, Photochem Photobiol 38: 487 (1983); Stryer, Annu Rev Biochem 47: 819-846 (1978); Fairclough et al., Methods Enzymol 48: 347-379 (1978)). When FRET is used as a contrast mechanism, co-localization of proteins and other molecules can be imaged with spatial resolution beyond the limits of conventional optical microscopy (Kenworthy, Methods 24: 289-296 (2001); Gordon et al., Biophys J 74: 2702-2713 (1998)).

Energy transfer efficiency is dependent upon a number of factors, including the transfer efficiency and the distance between the donor and acceptor (r). For example, the basic Forster energy transfer process involves the ability of a donor group to absorb photonic energy at one wavelength (hv₁) and transfer it, via a nonradiative process, to an acceptor group which re-emits the photonic energy at a longer wavelength (hv₂) or dissipates the energy nonradiatively. When the energy transfer is by nonradiative or Förster energy transfer, equations describing the relationship between efficiency of energy transfer and efficiency of energy transfer are known (for example, see Youvan et al., U.S. Pat. No. 6,456,734 and Heller, U.S. Pat. No. 6,416,953).

i) Förster Distance

The rate of energy transfer between the acceptor molecule and the donor molecule in FRET is inversely proportional to the sixth power of the distance between the donor and acceptor, thus, the energy transfer efficiency is extremely sensitive to distance changes. Energy transfer is said to occur with detectable efficiency in the 1-10 nm distance range. The distance at which energy transfer is 50% efficient (i.e., 50% of excited donors are deactivated by FRET) is defined by the Förster radius (Rₒ). The magnitude of Rₒ is dependent on the spectral properties of the donor and acceptor molecules and can be calculated from the spectral overlap integrals by using the equation:
Rₒ=[8.8×10²³·κ²·n⁻⁴·QY_{D}·J(λ)]^{1/6} angstrom
where κ²=dipole orientation factor (range 0 to 4; 78 ²=2/3 for randomly oriented donors and acceptors)

$$\begin{matrix}{QY}_{D} = fluorescence quantum yield \\ of the donor in the absence of the acceptor \\ n = refractive index \\ J ( λ ) = spectral overlap integral ( see below ) \\ = \int {ɛ}_{A} ( λ ) \cdot {F}_{D} ( λ ) \cdot {λ}^{4} ⅆ {cm}^{3} {M}^{- 1} where \\ {ɛ}_{A} = extinction coefficient of acceptor \\ {F}_{D} = fluorescence emission intensity of donor \\ as a fraction of the total integrated intensity\end{matrix}$$

The Förster distance must be considered in selecting the donor molecule and acceptor molecule of the energy transfer pair of the conjugate. The Forster distance also is considered in selecting the linking components or placement of the energy transfer pair, so that interaction of the targeting moiety with its target causes changes in the distance between the donor and acceptor molecules. These distances can be empirically determined or can be calculated. As a non-limiting example, the donor and acceptor molecule can be placed within about 1 to about 10 nm (10 angstrom to about 100 angstrom) to observe the energy transfer. Measurement of energy transfer involves monitoring a quenching of a signal from an excited energy donor, which decreases as the energy transfer compounds achieve proximity to one another.

iii) Selection Criteria

Fluorophores and/or quenchers for use as an energy transfer pair in the disclosed conjugate can be selected based on factors such as, but not limited to, cost, availability, size, absorption wavelength and emission wavelength. For example, because the conjugate is activated upon interaction of the targeting moiety to its target, use of certain fluorophore or quencher molecules can be precluded due to size or electrostatic constraints. In addition, photosensitizer and/or fluorophores and/or quenchers selected for use in the disclosed conjugates also must meet a variety of criteria to facilitate the energy transfer process. These criteria include, but are not limited to, acceptor-donor distance, overlap of donor emission and acceptor absorption, distinguishing of donor an acceptor peaks, quantum yield, and orientation of fluorophores.

a) Distance

As energy transfer reactions, for example, FRET, involve the transfer of energy from one luminescent molecule (i.e. the donor molecule) to another luminescent molecule (i.e. the acceptor molecule) in a distance-dependent manner, the donor and acceptor molecules must be placed in close proximity to facilitate energy transfer. As a non-limiting example, the donor and acceptor molecule can be placed within about 1 to about 10 nm to observe the energy transfer. One of skill in the art can vary the placement of the donor and acceptor molecules so that they are within the required proximity to transfer energy, so that that donor molecule is quenched. In particular, one of skill in the art can select the location and placement of the donor and acceptor molecules in the conjugate, run a sample FRET experiment to measure energy transfer, and adjust the placement of the donor and acceptor molecules until the donor molecule is quenched. Alternatively, one of skill in the art can use the literature, handbooks, manuals, internet, experimental results, and other sources well known in the art to determine the placement distance of the donor and acceptor molecules to achieve quenching of the donor molecule by the acceptor molecule.

When all other parameters are optimal, the efficiency of the energy transfer decreases as the distance between the donor and acceptor molecules increases, as 1/r⁶. For example, a donor to acceptor distance of less than 3.5 nm (35 Angstroms) can result in 50% efficient FRET energy transfer (see Heller, U.S. Pat No. 6,416,953). The conjugates disclosed herein are designed in such a way that when the conjugate is not interacting with a target, the donor molecule and acceptor molecule are positioned at a donor-acceptor transfer distance. In one embodiment, the donor and acceptor molecules are in close proximity so that quenching of the donor molecule is from about 25% to 100% efficient.

The optimum positioning or spacing of the donor molecule and the acceptor molecule to forming the donor-acceptor transfer distance can be determined empirically. In general, the conditions required for energy transfer, such as FRET are (i) that the donor and acceptor molecules be in close proximity to one another (typically 1 or 10 to 100 or 200 Angstroms) and (ii) that the absorption spectrum of the acceptor overlap the fluorescence emission spectrum of the donor.

b) Overlap of Donor Emission and Acceptor Absorption

A second criterion for determining a donor-acceptor energy transfer pair for use in the conjugates provided herein is that the energy emission spectrum of the donor molecule should at least partially overlap the energy absorption spectrum of the acceptor molecule, so that energy transfer from the donor to the acceptor can occur. Typically, an energy transfer donor compound has an emission peak wavelength (Dλₑₘ) that is within several nm of the excitation peak wavelength of the acceptor molecule (Aλₑₓ). The difference between Dλₑₘ and Aλₑₓ is typically from about 70 nm to about 20 nm or less. The difference between Dλₑₘ and Aλₑₓ can be about 60 nm, about 50 nm, about 30 nm, about 20 nm, about 15 nm, about 10 nm, about 5 nm, about 4 nm, about 3 nm, about 2 nm, or about 1 nm. In certain instances, the difference between the Dλₑₘ and Aλₑₓ can be larger than 70 nm (i.e., where light having a wavelength that is far from the excitation peak wavelength of the donor and/or the emission peak wavelength of the acceptor can be used) if the Dλₑₘ peak and the Aλₑₓ peak partially overlap and if the donor and acceptors are within proximity for detectable energy transfer to occur. Tables of spectral overlap integrals are readily available to those working in the field (for example, see Berlman, I. B., “Energy transfer parameters of aromatic compounds,” Academic Press, New York and London (1973)).

c) Limited Environmental Sensitivity

Another criterion that can be used to select the donor and acceptor molecules of the energy transfer pair is their sensitivity to assay or physiological conditions. As a non-limiting example, quenching agents that are unaffected by changes in pH, ion concentration, temperature, and solvent media can be selected for the conjugates described herein.

d) Quantum Yield

Energy transfer is most efficient when a donor fluorophore with high fluorescence quantum yield (such as one approaching 100%) is paired with an acceptor with a large extinction coefficient at wavelengths coinciding with the emission of the donor. Dependence of fluorescence energy transfer on the above parameters has been reported (Lakowicz, J. R., “Principles of Fluorescence Spectroscopy,” New York:Plenum Press (1983); and Herman, B., “Resonance energy transfer microscopy,” in: Fluorescence Microscopy of Living Cells in Culture, Part B, Methods in Cell Biology, Vol 30 (Taylor, D. L. & Wang, Y.-L., eds.), San Diego, Academic Press (1989), pp. 219-243).

e) Available Attachment Sites

Another factor to consider when selecting a donor molecule, such as a photosensitizing agent or a fluorophore, and an acceptor molecule, such as a quencher, is the available attachment sites. Most attachments are conveniently effected via sulfhydryl or amine interactions. Synthetic and commercial alternatives are available depending on the selected photosensitizing agent, fluorophore or quencher, the molecule or linking component used in the conjugate, and the environment in which it will reside. As noted above, the distance is selected so that interaction of the targeting moiety results in repositioning of the quenching agent out of a fluorescence-quenching interaction-permissive position. If the donor and acceptor molecules are too close, then interaction of the targeting agent may not end quenching of the donor molecule, since energy transfer would continue to occur. If the distance between the donor and accept molecule is too great, then energy transfer may not occur at all. The distances can be determined by any method, such as by calculation or empirically.

Techniques in synthetic chemistry provide methods for the attachment of donor molecules using a lining component that provides a donor-acceptor transfer distance (for example, see Heller et al., U.S. Pat. No. 4,996,143). For example, synthetic linkage techniques are known that allow incorporation of both a donor and an acceptor molecule within the same oligonucleotide (see Heller et al., U.S. Pat. No. 4,996,143). Using the particular linker arm, it was found that the most efficient energy transfer (in terms of re-emission by the acceptor) occurred when the donor and acceptor were spaced by 5 intervening nucleotide units, or approximately 2 nm apart. Heller et al., U.S. Pat. No. 4,996,143 also showed that as the nucleotide spacing was increased from 6 to 12 units (from about 2 nm to about 4 nm), the energy transfer efficiency was also found to decrease, which is in accordance with Förster theory. There is extensive guidance in the literature for derivatizing reporter and quencher molecules for covalent attachment via readily available reactive groups that can be added to a molecule. The diversity and utility of chemistries available for conjugating fluorophores to other molecules and surfaces is exemplified by the extensive body of literature on preparing nucleic acids derivatized with fluorophores. See, for example, Ullhman et al., U.S. Pat. No. 3,996,345 and Khanna et al., U.S. Pat. No. 4,351,760.

f) Modification of Energy Transfer

The components of the energy transfer pair to be used in the disclosed conjugate are generally selected so that an absorbance band of the acceptor molecule overlaps a fluorescence emission band of the donor molecule. Another factor to be considered in choosing the donor/acceptor energy transfer pair is the efficiency of energy transfer between them. The efficiency of energy transfer can easily be empirically tested using the methods known in the art. The efficiency of energy transfer between the donor-acceptor pair can be adjusted by changing the ability of the donor and acceptor to closely associate.

For example, an increase or decrease in association can be promoted by adjusting the length of a linking component between fluorophore or photosensitizing agent and the quenching agent. The ability of the donor-acceptor pair to associate also can be increased or decreased by adjusting the hydrophobic or ionic interactions or the steric repulsions between the two moieties in the disclosed conjugates. Thus, intramolecular interactions responsible for the association of the donor-acceptor pair can be enhanced or attenuated. Thus, for example, the association between the donor-acceptor pair can be increased by, for example, utilizing a donor bearing an overall negative charge and an acceptor with an overall positive charge.

2. Targeting Moiety

The conjugate disclosed herein includes a targeting moiety that preferentially associates or binds to a particular cell, tissue, receptor, infecting agent or an area of the body of the subject to be treated, such as a target cell, target tissue or target composition. The targeting moiety can be a polypeptide (which may be linear, branched, or cyclic). The targeting moiety can include a polypeptide having an affinity for a polysaccharide target, for example, a lectin (such as a seed, bean, root, bark, seaweed, fungal, bacterial, or invertebrate lectin). Particularly useful lectins include concanavalin A, which is obtained from jack beans, and lectins obtained from the lentil, Lens culinaris. The targeting moiety can be a molecule or a macromolecular structure (e.g. a liposome, a micelle, a lipid vesicle, or the like) that preferentially associates or binds to a particular tissue, receptor, infecting agent or other area of the body of the subject to be treated.

a. Exemplary Targeting Moieties

Examples of a targeting moiety include but are not limited to an oligonucleotide, a carbohydrate, a carbohydrate polymer (such as dextran sulfate or heparin), a receptor, a ligand and one member of a ligand-receptor binding pair. The ligands useful as a targeting moiety include those that are receptor-specific as well as immunoglobulins and fragments thereof For example, immunoglobulins useful as targeting moieties include antibodies in general and monoclonal antibodies, as well as immunologically reactive fragments thereof.

For example, the following receptors can be used to target macrophages: the complement receptor (Rieu et al., J. Cell Biol. 127:2081-2091, 1994), the scavenger receptor (Brasseur et al., Photochem. Photobiol. 69:345-352, 1999, the transferrin receptor (Dreier et al., Bioconjug. Chem. 9:482-489, 1998; Hamblin et al., J. Photochem. Photobiol. 26:4556, 1994); the Fc receptor (Rojanasakul et al., Pharm. Res. 11:1731-1733, 1994); the mannose receptor (Frankel et al., Carbohydr. Res. 300:251-258, 1997; Chakrabarty et al., J. Protozool. 37:358-364, 1990). Targeting moieties that can be conjugated with photosensitizers, for example to target to macrophages, include low density lipoproteins (Mankertz et al., Biochem. Biophys. Res. Commun. 240:112-115, 1997; von Baeyer et al., Int. J. Clin. Pharmacol. Ther. Toxicol. 31:382-386, 1993), very low density lipoproteins (Tabas et al., J. Cell Biol. 115:1547-1560, 1991), mannose residues and other carbohydrate moieties (Pittet et al., Nucl. Med. Biol. 22:355-365, 1995), poly-cationic molecules, such as poly-L-lysine (Hamblin et al., J. Photochem. Photobiol. 26:45-56, 1994), liposomes (Bakker-Woudenberg et al., J. Drug Target. 2:363-371, 1994; Betageri et al., J. Pharm. Pharmacol. 45:48-53, 1993), antibodies (Gruenheid et al., J. Exp. Med. 185:717-730, 1997), and 2-macroglobulin (Chu et al., J. Immunol. 152:1538-1545, 1994).

Many targeting moieties and methods for targeting compounds are well known to those of skill in the art. All such targeting methods are contemplated herein for use in the instant conjugates. For non-limiting examples of targeting methods, see, e.g., U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542 and 5,709,874.

b. Placement

The donor molecule and an acceptor molecule of the conjugate disclosed herein are positioned to be in a donor-acceptor transfer distance so that the conjugate is in a non-reactive state when it is not interacting with target. When the conjugate interacts with a target cell or target tissue via the targeting moiety, the donor molecule and the acceptor molecule are separated such that energy transfer between them no longer occurs. Thus, the spatial rearrangement of the donor molecule and acceptor molecule in the conjugate occurs only after interaction of the targeting moiety with its target. Hence, the targeting moiety is selected and positioned in the conjugate so that when the targeting moiety interacts with its target, the spatial arrangement of the conjugate is changed such that the donor molecule and acceptor molecule are no longer in a donor-acceptor transfer distance.

For example, in one embodiment, upon binding of the conjugate to its target, the three dimensional structure of the conjugate is altered in such a way that the quenching agent is no longer positioned close enough to quench the excited state of the photosensitizer—thus allowing the photosensitizer to function as required for fluorescence-based diagnostic methods or for PDT by generation of singlet oxygen (¹O₂). In the latter case, the singlet oxygen is then available to destroy the target. When this embodiment is used for diagnostic purposes, the photosensitizer need only function as a fluorophore. The quenching agent of this invention then serves to prevent the generation of false positive signals from the fluorophore when it is not bound to the target.

In another embodiment, the donor molecule is a porphyrin or porphyrin derivative tetrapyrrole that includes a metal atom in its central coordination cavity and the acceptor molecule is a quenching agent with one or more suitable functional groups that coordinate to the axial position of the metal coordinated within the photosensitizing agent. The targeting moiety is positioned in the conjugate in such a way that the interaction of the targeting moiety with the target disrupts the association of the axial ligand to the metal, releasing the quenching agent and allowing the porphyrin or porphyrin derivative tetrapyrrole to be activated when irradiated.

C. Preparation of the Conjugates

The conjugates provided herein may be prepared according to methods known so those skilled in the art, for example as provided below and exemplified herein (see, e.g., EXAMPLES 1 through 3).

1. Coupling Agents

Techniques to construct conjugates of ligands with photosensitizers are well known to those of ordinary skill in this art. For example, Rakestraw et al. teaches conjugating Sn(IV) chlorin e via covalent bonds to monoclonal antibodies using a modified dextran carrier (Rakestraw, S. L., Tompkins, R. D., and Yarmush, M. L., Proc. Nat. Acad. Sci. USA 87: 4217-4221 (1990). The conjugates disclosed herein can be conjugated to a ligand, such as an antibody, by using a coupling agent. Any bond which is capable of linking the components such that they are stable under physiological conditions for the time needed for administration and treatment is suitable, but covalent linkages are preferred. The link between two components may be direct, e.g., where a photosensitizer is linked directly to a targeting agent, or indirect, e.g., where a photosensitizer is linked to a linking component and that linking component being linked to the targeting agent.

A coupling agent should function under conditions of temperature, pH, salt, solvent system, and other reactants that substantially retain the chemical stability of the donor molecule, the acceptor molecule and the targeting moiety. Coupling agents should link component moieties stably, but such that there is only minimal or no denaturation or deactivation of the donor molecule, acceptor molecule or the targeting moiety. Many coupling agents react with an amine and a carboxylate, to form an amide, or an alcohol and a carboxylate to form an ester. Coupling agents are known in the art (see, e.g., M. Bodansky, “Principles of Peptide Synthesis”, 2nd ed., and T. Greene and P. Wuts, “Protective Groups in Organic Synthesis,” 2nd Ed, 1991, John Wiley, NY). Representative combinations of such groups are amino with carboxyl to form amide linkages, or carboxy with hydroxy to form ester linkages or amino with alkyl halides to form alkylamine linkages, or thiols with thiols to form disulfides, or thiols with maleimides or alkyl halides to form thioethers. Obviously, hydroxyl, carboxyl, amino and other functionalities, where not present may be introduced by known methods.

The conjugates provided herein can be prepared by coupling the photosensitizer to a targeting moiety, such as an antibody for example, by cleaving an available ester moiety on the photosensitizer and coupling the compound via peptide linkages to an antibody through an N terminus, or by other methods known in the art. A variety of coupling agents, including cross-linking agents, can be used for covalent conjugation. Examples of cross-linking agents include N,N′-dicyclohexylcarbodiimide (DCC), N-succinimidyl-S-acetyl-thioacetate (SATA), N-succinimidyl-3-(2-pyridyldithio)propionate (SPDP), ortho-phenylene-dimaleimide (o-PDM), and sulfosuccinimidyl 4N-maleimido-methyl)-cyclohexane-1-carboxylate (sulfo-SMCC). See, e.g., Karpovsky et al. J Exp. Med. 160:1686 (1984); and Liu, M A et al., Proc. Natl. Acad. Sci. USA 82: 8648 (1985). Other methods include those described by Brennan et al. Science 229: 81-83 (1985) and Glennie et al., J. Immunol. 139: 2367-2375 (1987). A large number of coupling agents for peptides and proteins, along with buffers, solvents, and methods of use, are described in the Pierce Chemical Co. catalog, pages O-90 to O-110 (1995, Pierce Chemical Co., 3747 N. Meridian Rd., Rockford Ill., 61105, U.S.A.), which catalog is hereby incorporated by reference.

For example, DCC is a useful coupling agent that can be used to promote coupling of the alcohol NHS to chlorin e6 in DMSO forming an activated ester which can be cross-linked to polylysine. DCC is a carboxy-reactive cross-linker commonly used as a coupling agent in peptide synthesis, and has a molecular weight of 206.32. Another useful cross-linking agent is SPDP, a heterobifunctional cross-linker for use with primary amines and sulfhydryl groups. SPDP has a molecular weight of 312.4, a spacer arm length of 6.8 angstroms, is reactive to NHS-esters and pyridyldithio groups, and produces cleavable cross-linking such that, upon further reaction, the agent is eliminated so the photosensitizer can be linked directly to a linking component or targeting agent. Other useful conjugating agents are SATA for introduction of blocked SH groups for two-step cross-linking, which is deblocked with hydroxylamine-HCl, and sulfo-SMCC, reactive towards amines and sulfhydryls. Other cross-linking and coupling agents are also available from Pierce Chemical Co. Additional compounds and processes, particularly those involving a Schiff base as an intermediate, for conjugation of proteins to other proteins or to other compositions, for example to reporter groups or to chelators for metal ion labeling of a protein, are disclosed in EPO 243,929 A2 (published Nov. 4, 1987).

2. Reactive Groups

The acceptor molecule or targeting moiety can be conjugated, directly or through a linking component, to the donor molecule using reactive groups, either on the donor molecule or on the acceptor molecule or the targeting moiety. For example, molecules that contain carboxyl groups can be joined to lysine □-amino groups in the target polypeptides either by preformed reactive esters (such as N-hydroxy succinimide ester) or esters conjugated in situ by a carbodiimide-mediated reaction. The same applies to molecules that contain sulfonic acid groups, which can be transformed to sulfonyl chlorides which react with amino groups. Molecules that have carboxyl groups can be joined to amino groups, such as on a polypeptide, by an in situ carbodiimide method. Molecules can also be attached to hydroxyl groups of serine or threonine residues or to sulfhydryl groups of cysteine residues.

Methods of joining components of a conjugate, e.g., coupling polyamino acid chains bearing photosensitizers to antibacterial polypeptides, can use heterobifunctional cross linking reagents. These agents bind a functional group in one chain and to a different functional group in the second chain. These functional groups typically are amino, carboxyl, sulfhydryl, and aldehyde. There are many permutations of appropriate moieties which will react with these groups and with differently formulated structures, to conjugate them together. See the Pierce Catalog, and Merrifield, R. B. et al., Ciba Found Symp. 186: 5-20 (1994).

The photosensitizer component of the conjugate may be optionally functionalized so as to include a liking component which allows the photosensitizer component to be linked to a targeting moiety, such as an analyte, antigen, antibody or other molecule. For example, the linking component may include, but is not limited to, an oligonucleotide, a polynucleotide, a nucleic acid, an oligosaccharide, a polysaccharide or an □,□-diaminoalkane linking species, such as 1,3-diaminopropane. A variety of linking components which are suited to this purpose have been described. For example, see Kricka, J. J., “Ligand-Binder Assays; Labels and Analytical Strategies,” pages 15-51 (Marcel Dekker, Inc., New York, N.Y. (1985)). The photosensitizer component is linked to the linking component and the linking component is linked to the analyte, antigen, antibody or other molecule using conventional techniques.

a. Exemplary Reactive Groups and Reactions

Reactive groups and classes of reactions useful in preparing the disclosed conjugates are generally those that are well known in the art of bioconjugate chemistry. Classes of reactions include those that proceed under relatively mild conditions. These include, but are not limited to nucleophilic substitutions (e.g., reactions of amines and alcohols with acyl halides, active esters), electrophilic substitutions (e.g. enamine reactions) and additions to carbon-carbon and carbon-heteroatom multiple bonds (e.g., Michael reaction). These and other useful reactions are discussed in, for example, Morrison et al., “Organic Chemistry,” 4th Ed., Allyn and Bacon, Inc., 1983, and Hermanson, “Bioconjugate Techniques,” Academic Press, San Diego, 1996.

For example, useful reactive functional groups include:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups, which can be converted to esters, ethers, aldehydes, etc.

(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups, which are capable of participating in Diels-Alder reactions such as, for example, maleimido groups;

(e) carbonyl groups, such that subsequent derivatization is possible via formation of carbonyl derivatives such as, for example, imines, hydrazones, semicarbazones or oximes, or via such mechanisms as Grignard addition or alkyllithium addition;

(f) sulfonyl groups for subsequent reaction with amines, for example, to form sulfonamides;

(g) thiol groups, which can be converted to disulfides or reacted with acyl halides;

(h) amine or sulfhydryl groups, which can be, for example, acylated, alkylated or oxidized;

(i) alkenes, which can undergo, for example, cycloadditions, acylation, Michael addition, etc;

(j) epoxides, which can react with, for example, amines and hydroxyl compounds; and

(k) phosphoramidites and other standard functional groups useful in nucleic acid synthesis.

There is extensive guidance in the literature for derivatizing photosensitizer and quencher molecules for covalent attachment via readily available reactive groups that can be added to a molecule. The diversity and utility of chemistries available for conjugating fluorophores, including photosensitizers, to other molecules and surfaces is exemplified by the extensive body of literature on preparing nucleic acids derivatized with fluorophores. See, for example, Ullman et al., U.S. Pat. No. 3,996,345 and Khanna et al., U.S. Pat. No. 4,351,760.

D. Pharmaceutical Compositions

1. Formulation of Pharmaceutical Compositions

The pharmaceutical compositions provided herein contain therapeutically effective amounts of one or more of the conjugates provided herein that are useful in the prevention, treatment, or amelioration of one or more of the symptoms of diseases or disorders associated with hyperproliferating tissue or neovascularization, or in which hyperproliferating tissue or neovascularization is implicated, in a pharmaceutically acceptable carrier. Diseases or disorders associated with hyperproliferating tissue or neovascularization include, but are not limited to, a cancer or a carcinoma, a tumor, acute glomerulonephritis, membrano-proliferative glomerulonephritis, myelomas, psoriasis, atherosclerosis, psoriatic arthritis, rheumatoid arthritis, diabetic retinopathies, macular degeneration, corneal neovascularization and choroidal hemangioma Pharmaceutical carriers suitable for administration of the conjugates provided herein include any such carriers known to those skilled in the art to be suitable for the particular mode of administration.

In addition, the compositions may be formulated as the sole pharmaceutically active ingredient in the composition or may be combined with other active ingredients.

The pharmaceutical formulations include one or more conjugates provided herein. The compositions are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the conjugates described above are formulated into pharmaceutical compositions using techniques and procedures well known in the art (see, e.g. Ansel, “Introduction to Pharmaceutical Dosage Forms,” 4th Ed. 1985, page 126).

Effective concentrations of one or more conjugates or pharmaceutically acceptable derivatives thereof is (are) mixed with a suitable pharmaceutical carrier. The conjugates may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats, prevents, or ameliorates one or more of the symptoms of diseases or disorders associated with hyperproliferating tissue or neovascularization or in which hyperproliferating tissue or neovascularization is implicated.

In one embodiment, the conjugates disclosed herein are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

The composition is included in a pharmaceutically acceptable carrier in an amount sufficient to exert a therapeutically useful effect in the absence of undesirable side effects on the patient treated. The therapeutically effective concentration may be determined empirically by testing the compositions in in vitro and in vivo systems known in the art, for example as described in U.S. Pat. No. 5,952,366 to Pandey et al (1999) and then extrapolated therefrom for dosages for humans.

The concentration of the pharmaceutical composition will depend on absorption, inactivation and excretion rates of the active composition, the physicochemical characteristics of the composition, the dosage schedule, and amount administered as well as other factors known to those of skill in the art. For example, the amount that is delivered is sufficient to ameliorate one or more of the symptoms of diseases or disorders associated with hyperproliferating tissue or neovascularization or in which hyperproliferating tissue or neovascularization is implicated.

In one embodiment, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the conjugates provided herein.

In instances in which the compositions exhibit insufficient solubility, methods for solubilizing the compositions may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the composition(s), the resulting mixture may be a solution, suspension, emulsion or the like. The form of the resulting mixture depends upon a number of factors, including the intended mode of administration and the solubility of the composition in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the conjugates or pharmaceutically acceptable derivatives thereof. The pharmaceutically therapeutically active compositions are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active composition sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active composition as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see “Remington's Pharmaceutical Sciences” (Mack Publishing Company, Easton, Pa., 15th Edition, 1975).

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, for example, in one embodiment 0.1-95%, and in another embodiment 75-85%.

2. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

a. Solid Compositions for Oral Administration

In certain embodiments, the formulations are solid dosage forms; in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, xanthan gum, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, gellan gum, sodium carboxymethylcellulose, polyethylene glycol 4000 and cellulose acetate phthalate.

The conjugate, or pharmaceutically acceptable derivative thereof could be provided in a composition that protects it from the acidic environment of the stomach. For example, the composition can be formulated in an enteric coating that maintains its integrity in the stomach and releases the active composition in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compositions can also be administered as a component of an elixir, suspension, syrup, wafer, sprinkle, chewing gum or the like. A syrup may contain, in addition to the active compositions, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a conjugate or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

b. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, xanthan gum, Veegun clay and acacia Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active composition or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a conjugate provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, ethanolamine, hydroxycoumarins, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

3. Injectables, Solutions and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a conjugate provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The composition diffuses through the outer polymeric membrane in a release rate controlling step. The percentage of active composition contained in such parenteral compositions is highly dependent on the specific nature thereof, as well as the activity of the composition and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, xanthan gum, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active composition is adjusted so that an injection provides an effective amount to produce the desired pharmacological or therapeutic effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active composition is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active composition to the treated tissue(s).

The composition may be suspended in micronized or other suitable form or may be derivatized to produce a more soluble active product or to produce a prodrug. The form of the resulting mixture depends upon a number of factors, including the intended mode of administration and the solubility of the composition in the selected carrier or vehicle. The effective concentration is sufficient for ameliorating the symptoms of the condition and may be empirically determined.

4. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a conjugate provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the composition. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected composition. Such amount can be empirically determined.

5. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The conjugates or pharmaceutically acceptable derivatives thereof may be formulated as aerosols for topical application, such as by inhalation (see, e.g., U.S. Pat. Nos. 4,044,126; 4,414,209; and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, and in another embodiment have diameters of less than 10 microns.

The compositions may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active composition alone or in combination with other pharmaceutically acceptable excipients can also be administered. These solutions, particularly those intended for ophthalmic use, may be formulated as 0.01%-10% isotonic solutions, pH about 5-7, with appropriate salts.

6. Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983; 6,261,595; 6,256,533; 6,167,301; 6,024,975; 6,010715; 5,985,317; 5,983,134; 5,948,433 and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

E. Articles of Manufacture

The compositions or pharmaceutically acceptable derivatives thereof may be packaged as articles of manufacture containing packaging material, a conjugate or pharmaceutically acceptable derivative thereof provided herein, which is effective for modulating the activity of hyperproliferating tissue or neovascularization, or for treatment, prevention or amelioration of one or more symptoms of hyperproliferating tissue or neovascularization mediated diseases or disorders, or diseases or disorders in which hyperproliferating tissue or neovascularization activity, is implicated, within the packaging material, and a label that indicates that the conjugate, or pharmaceutically acceptable derivative thereof, is used for modulating the activity of hyperproliferating tissue or neovascularization, or for treatment, prevention or amelioration of one or more symptoms of hyperproliferating tissue or neovascularization mediated diseases or disorders, or diseases or disorders in which hyperproliferating tissue or neovascularization is implicated.

The articles of manufacture provided herein contain packaging materials. Packaging materials for use in packaging pharmaceutical products are well known to those of skill in the art. See, e.g., U.S. Pat. Nos. 5,323,907; 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the conjugates and compositions provided herein are contemplated as are a variety of treatments for any disease or disorder in which hyperproliferating tissue or neovascularization is implicated as a mediator or contributor to the symptoms or cause.

F. Kits

Any one of the conjugates disclosed herein or a pharmaceutically acceptable derivative thereof may be supplied in a kit along with instructions on conducting any of the methods disclosed herein. Instructions may be in any tangible form, such as printed paper, a computer disk that instructs a person how to conduct the method, a video cassette or digital video device containing instructions on how to conduct the method, or computer memory that receives data from a remote location and illustrates or otherwise provides the instructions to a person (such as over the Internet).

Further provided are kits for detecting a target tissue or target composition, such as in a sample, or for diagnosing an infecting agent, the kits including any one of the conjugates described herein that includes a targeting moiety that targets the target tissue or target composition, and instructions, for example, for carrying out assays or for interpreting results or for aid in determining if a target tissue is present in a subject, or if a subject is infected with an infecting agent. The kits also optionally contain one or more containers (microtitre trays, eppendorf tubes, etc.) for holding the conjugates or for performing an assay. The kits can also include standards for calibrating any detection reaction or assay using the conjugates.

G. Methods of use of the Conjugates

1. Methods of PDT, Diagnostic and Therapeutic Applications

Briefly, the composition is generally administered to the subject before the target tissue, target composition or subject is subjected to illumination. The composition is administered as described elsewhere herein.

The dose of a conjugate disclosed herein for an optimal therapeutic level can be determined clinically. A certain length of time is allowed to pass for the circulating or locally delivered conjugate to be taken up by the target tissue. The unbound conjugate is cleared from the circulation during this waiting period, or additional time can optionally be provided for clearing of the unbound conjugate from non-target tissue. The waiting period will be determined clinically and may vary depending on the composition of the composition.

At the conclusion of this waiting period, a light source is used to activate the bound conjugate. The light source may provide non-coherent (non-laser) or coherent (laser) light. For example, non-coherent light sources include, but are not limited to, mercury or xenon arc lamps with optical filters, tungsten lamps, cold cathode fluorescent lamps, halogen lamps, light emitting diodes (LEDs), LED arrays, incandescent sources, and other electroluminescent devices. Lamp sources are used when fine definition of the illumination region is not required, or when a large region is to be illuminated. Focused non-coherent light can be used to illuminate small regions, such as by using lenses to focus the light or optical fibers to direct or deliver the light. Laser sources are usually used to illuminate small, well-defined regions, because of their higher specific radiance and more readily controlled beam properties. Coherent light sources include, but are not limited to, dye lasers, argon ion lasers, laser diodes, tunable lasers, Ti-sapphire lasers, Ruby lasers, Alexandrite lasers, Helium-Neon lasers, GaAlAs and InGaAs diode lasers, Nd-YLF lasers, Nd-glass lasers, Nd-YAG lasers and fiber lasers. For example, lasers are often used as excitation sources in confocal equipment, and to create very high flux. Laser sources are limited in that they emit a restricted, often discrete set of wavelengths in contrast to lamps, which generally produce a continuous spectrum that can be filtered to provide any desired band within a certain range.

The area of illumination is determined by the location and dimension of the pathologic region to be detected, diagnosed or treated. The duration of illumination period will depend on whether detection or treatment is being performed, and can be determined empirically. A total or cumulative period of time anywhere from between about 1 minute and 72 hours can be used. In one embodiment, the illumination period is between about 4 minutes and 48 hours. In another embodiment, the illumination period is between about 30 minutes and 24 hours.

The total fluence or energy of the light used for irradiating is between about 10 Joules and about 25,000 Joules; in some embodiments, the total fluence is between about 100 Joules and about 20,000 Joules or between about 500 Joules and about 10,000 Joules. Light of a wavelength and fluence sufficient to produce the desired effect is selected, whether for detection by fluorescence or for therapeutic treatment to destroy or impair a target tissue or target composition. Light having a wavelength corresponding at least in part with the characteristic light absorption wavelength of the photosensitizing agent is used for irradiating the target issue.

The power delivered by the light used is measured in watts, where 1 watt is equal to 1 joule/sec. Intensity is the power per area. Thus, intensity may be measured in watts/cm². Therefore, the intensity of the light used for irradiating in the present invention may be between about 5 mW/cm² to about 500 mW/cm². Since the total fluence or amount of energy of the light in Joules is divided by the duration of total exposure time in seconds, the longer the amount of time the target is exposed to the irradiation, the greater the amount of total energy or fluence may be used without increasing the amount of the intensity of the light used. The present invention employs an amount of total fluence of irradiation that is sufficiently high to activate a conjugate disclosed herein.

In one embodiment of using the conjugates disclosed herein for photodynamic therapy, a conjugate is injected into the mammal, e.g. human, to be diagnosed or treated. The level of injection is usually between about 0.1 and about 0.5 μmol/kg of body weight. In the case of treatment, the area to be treated is exposed to light at the desired wavelength and energy, e.g. from about 10 to 200 J/cm². In the case of detection, fluorescence is determined upon exposure to light at a wavelength sufficient to cause the conjugate to fluoresce at a wavelength different than that used to illuminate the conjugate. The energy used in detection is sufficient to cause fluorescence and is usually significantly lower than is required for treatment.

2. Detecting Target Tissue or Target Compositions

In addition to PDT, the compositions provided herein can be used to detect target cells, target tissue, or target compositions in a subject. When one of the conjugates provided herein is to be used for detection of a target tissue or a target composition, the conjugate is introduced into the subject and sufficient time is allowed for the conjugate to accumulate in the target tissue or to become associated with the target composition. The area of treatment is then irradiated, generally using light of an energy sufficient to cause fluorescence of the conjugate, and the energy used is usually significantly lower than is required for photodynamic therapy treatment. Fluorescence is determined upon exposure to light at the desired wavelength, and the amount of fluorescence can be correlated to the presence of the conjugate, qualitatively or quantitatively, by methods known in the art.

3. Diagnosing an Infecting Agent

The conjugates provided herein can be used to diagnose the presence of an infecting agent, or the identity of an infecting agent in a subject. In this embodiment, the targeting moiety of the conjugates provided herein is selected to be specific for an infecting agent. For example, the selected targeting moiety can be an antibody or antibody fragment that selectively associates with the infecting agent, and after allowing sufficient time for the disclosed conjugate to associate with the infecting agent and to clear from non-target tissue, the conjugate can be visualized, such as by exposing to light of an energy sufficient to cause fluorescence of the conjugate. By way of example, any one of the conjugates provided herein can include as a targeting moiety an antibody that is targeted against a suitable Helicobacter pylori antigen. The conjugate is formulated into a pharmaceutical preparation that, when introduced into a subject, releases the conjugate to a gastric mucus/epithelial layer where the bacterium is found. After sufficient time for the conjugate to selectively associate with the target infecting agent, and for any unbound conjugate to clear from non-target tissue, the subject can be examined to determine whether any Helicobacter pylori is present. This can be done, for example, by irradiating the suspect target area with light of an energy sufficient to cause fluorescence of the conjugate, such as by using fiberoptics, and detecting any fluorescence of the conjugate.

4. Fluorescence Immunoassays

One problem that has plagued fluorescence immunoassays has been discriminating the fluorescent signal of interest from background radiation. The intensity of signal from background radiation may be up to 10,000 times larger than the intensity of the fluorescent signal of interest. The problem of background detection is particularly pronounced in assays of biological samples. For example, in the analysis of blood plasma, the presence of a naturally occurring fluorescable material, biliverdin, causes substantial background radiation. Such compounds are highly fluorescent and contribute significant background signals which interfere with the label's signal, thus limiting the sensitivity of assays using fluorescein labels.

When any one of the disclosed conjugates is used for diagnostic purposes, the photosensitizer component need only function as a fluorophore. The quenching agent of this embodiment then serves to prevent the generation of false positive signals from the fluorophore when it is not bound to the target. It is only upon interaction of the targeting moiety of the disclosed conjugate with the target cell, target tissue or target composition that the quenching agent is moved out of a fluorescence-quenching interaction-permissive position with the photosensitizing agent that the photosensitizing agent can function as a fluorophore.

Fluorescent immunoassays are well known to those in this art. For example, in one embodiment, a sample can be analyzed for the presence of an infecting agent or a target composition. The sample can be fixed to a solid support or the assay can be done in solution. The disclosed conjugate is added to and incubated with the sample under biological assay conditions. If the test sample is fixed to a solid support, excess unbound conjugate optionally can be removed, such as by washing the solid support with buffer, saline, or distilled water. Because of the nature of the conjugates disclosed herein, only conjugate bound to the target via the targeting moiety will fluoresce when illuminated. Detection and measurement of the conjugate that is bound to the sample being analyzed results in a value that may be compared to a comparative value for qualitative or quantitative determinations.

The following examples are included for illustrative purposes only and are not intended to limit the scope of the invention.

### EXAMPLES

### Example 1

A photosensitizer, such as Talaporfin Sodium, is conjugated with a covalent linkage to one end of a single-stranded oligonucleotide. The oligonucleotide begins and ends with mutually complementary sequences with the remainder of the oligonucleotide in between consisting of a binding sequence, known to have a suitable degree of binding affinity for the target tissue or structure. The opposite end of the oligonucleotide is conjugated via a covalent linkage to a non-fluorescent quenching agent.

A therapeutically useful amount of this conjugate is administered to the subject. After a sufficient time for the agent to bind to the intended target and clear from normal tissue, a light source of the appropriate wavelength is used to deliver a therapeutically useful amount of light to an area that includes the lesion or region of hyperproliferative tissue.

### Example 2

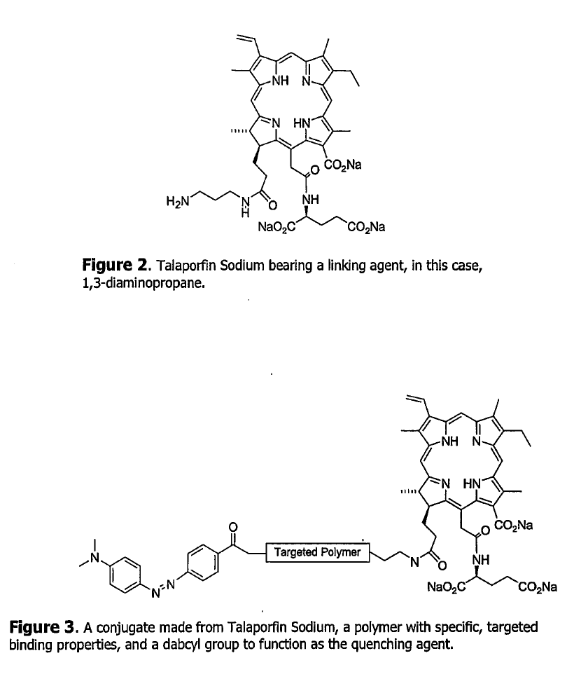

In another embodiment, the photosensitizer Talaporfin Sodium is derivatized, using a water-soluble carbodiimide reagent, with a commercially available α,ω-diaminoalkane linking species, such as 1,3-diaminopropane, to afford the monoamino compound shown in FIG. 2. This species is then linked via a sulfhydryl-reactive linking moiety on a targeting moiety, such as an antibody or a polymer that demonstrates selective targeting in biological systems, such as an oligonucleotide or oligopeptide, using methods known in the art. These usually include treating with an electrophile (such as a haloacetyl or a maleiimidyl group) that reacts chemically with a thiol function. The single-amino structure of the species depicted in FIG. 2 allows for the preparation of regiochemically-defined species in which the quenching agent is covalently linked to the remainder of the composition. One of ordinary skill in the art can use this method to link the quenching agent to oligonucleotides obtained in commercially available form in which a sulfhydryl-terminated alkyl group is located on the 5′ phosphate.

### Example 3

A photosensitizer, such as Talaporfin Sodium is conjugated, via amide linkage, to one terminus of a polymer known to exhibit selective binding to the target. The opposite end of the polymer is conjugated to a quenching agent such as a dabcyl (4-(4′-dimethylaminophenylazo)benzoyl) group, by reaction with a commercially available agent such as dabcyl chloride. This agent can be further modified by the addition of a suitable metal ion to an aqueous solution of the composition. The metal binds to the coordination pocket of the porphyrin ring-system and also coordinates the amine or azo group of the quenching group, ensuring that the quenching agent remains sufficiently close to the photosensitizer to allow energy transfer and thereby quench the generation of singlet oxygen. Binding of the targeted polymer to its target then disrupts this coordination binding environment, releasing the quenching agent from the metal and allowing the quenching agent to move away from the photosensitizer and restoring its activity.

A therapeutically useful amount of this exemplary conjugate is administered to the subject. After a sufficient time for the conjugate to bind to the intended target and clear from normal tissue, a light source of the appropriate wavelength is used to deliver a therapeutically useful amount to light to an area that includes the lesion or region of hyperproliferative tissue.

Since modifications will be apparent to those of skill in this art, it is intended that this invention be limited only by the scope of the appended claims. All patents, published patent applications and non-patent documents referred to herein are hereby incorporated by reference.

What is claimed is:

1. A conjugate, comprising:
a fluorophore or a photosensitizer
a quenching agent; and
a targeting moiety, wherein:
the fluorophore or the photosensitizer is linked to
the quenching agent and the targeting moiety in such a way that activation of the fluorophore or the photosensitizer is quenched until the targeting moiety is bound to a target, whereupon the quenching agent moves away from the photosensitizer, enabling activation of the photosensitizer upon irradiation with light of a suitable wavelength.

2. The conjugate of claim 1, wherein the fluorophore is 5-((2-aminoethyl)-amino)naphthalene-l-sulfonic acid (EDANS).

3. The conjugate of claim 1, wherein the photosensitizer is a porphyrin.

4. The conjugate of claim 1, wherein the photosensitizer is a chlorin.

5. The conjugate of claim 1, wherein the photosensitizer is a bacteriochlorin.

6. The conjugate of claim 1, wherein the quenching agent is □-carotene or a derivative thereof.

7. The conjugate of claim 1, wherein the targeting moiety is an antibody.

8. The conjugate of claim 1, wherein the targeting moiety is a fragment or other derivative of an antibody.

9. The conjugate of claim 1, wherein the targeting moiety is selected from the group consisting of an antigen, a ligand, a receptor, one member of a specific binding pair, a polyamide, a peptide, an oligosaccharide, a polysaccharide, a low density lipoprotein (LDL) or an apoprotein of LDL, a steroid, a steroid derivative, a hormone and a hormone-mimic.

10. The conjugate of claim 1, wherein the photosensitizer and the quenching agent include a linking component to link with an amino or hydroxy fatty acid or sulfonic acid of from 1 to 20 carbon atoms using ester, amide, or sulfonamide linkages.

11. The conjugate of claim 10, wherein the linking component is an oligonucleotide of 20-60 residues.

12. The conjugate of claim 11, wherein the oligonucleotide contains a specific sequence for binding to a desired target, along with at least one pair of mutually complementary regions that cause it to adopt a conformation, in the absence of the target, in which the quenching agent is sufficiently near to the photosensitizer to render the photosensitizer inactive; wherein binding of the target-specific sequence to the target disrupts the conformation, allowing the photosensitizer to become active upon illumination with light of an appropriate wavelength.

13. The conjugate of claim 1, wherein the quenching agent is 4-(4′-dimethylamino-phenylazo)benzoic acid (DABCYL) or 4-(4′dimethylamino-phenylazo)sulfonic (DABSYL).

14. The conjugate of claim 1, wherein the photosensitizer and quenching agent are linked by a polymer that exhibits binding specificity for the desired target, wherein in the absence of the target, the linked system adopts a conformation in which the quenching agent is sufficiently close to the photosensitizer to render it photochemically inert and wherein in the presence of the target, this conformation is disrupted, enabling photochemical processes to be carried out.

15. The conjugate of claim 1, wherein the photosensitizer comprises a porphyrin or porphyrin derivative tetrapyrrole and bears a physiologically acceptable metal atom in its central coordination cavity and one or more suitable functional groups are located on or near the quenching agent that efficiently coordinate to the axial position of the metal coordinated within the photosensitizer; and the targeting moiety is located in such a way that the presence of the target disrupts the relatively weak association of the axial ligand to the metal, releasing the quenching agent and rendering the fluorescent or PDT agent active.

16. The conjugate of claim 1, wherein the fluorophore emits light of a suitable wavelength for excitation of more than one type of secondary fluorophore or photosensitizer and the presence of more than one secondary fluorophore or photosensitizer allows the composition to produce different responses, designed to be appropriate to the presence of each of the targets with which the composition can react.

17. The conjugate of claim 1, wherein the targeting moiety is a polymer bearing at least one sulfate or sulfonate functional group.

18. The conjugate of claim 17, wherein the targeting moiety is dextran sulfate.

19. The conjugate of claim 18, wherein the dextran sulfate has an average molecular weight of about 5,000.

20. The conjugate of claim 1, wherein the photosensitizer is Talaporfin sodium.

21. A pharmaceutical composition, comprising the conjugate of claim 1 or a pharmaceutically acceptable derivative thereof in a pharmaceutically acceptable carrier.

22. An article of manufacture, comprising:
packaging material; and
the conjugate of claim 1 or a pharmaceutically acceptable derivative thereof contained within the packaging material, wherein:
the conjugate or pharmaceutically acceptable derivative thereof is effective in a photodynamic therapy treatment for ameliorating the symptoms of a hyperproliferative disorder; and
the packaging material includes a label that indicates that the composition or salt thereof is used in a photodynamic therapy treatment for ameliorating the symptoms of a hyperproliferative disorder.

23. A method for administering a photodynamic therapy to a target, comprising:
(i) administering to a subject a conjugate of claim 1 or a pharmaceutically acceptable derivative thereof that preferentially associates with the target; and
(ii) irradiating the subject with light of a wavelength and total fluence sufficient to produce a therapeutic effect.

24. The method of claim 23, wherein the target is selected from the group consisting of a vascular endothelial tissue, a neovasculature tissue, a neovasculature tissue present in an eye, an abnormal vascular wall of a tumor, a solid tumor, a tumor of a head, a tumor of a neck, a tumor of an eye, a tumor of a gastrointestinal tract, a tumor of a liver, a tumor of a breast, a tumor of a prostate, a tumors of a lung, a nonsolid tumor, malignant cells of one of a hematopoietic tissue and a lymphoid tissue, lesions in a vascular system, a diseased bone marrow, and diseased cells in which the disease is one of an autoimmune and an inflammatory disease.

25. The method of claim 24, wherein the target composition is selected from the group consisting of bacteria, viruses, fungi, protozoa, and toxins.

26. The method of claim 24, further comprising the step of allowing sufficient time for any of the conjugate that is not preferentially associated to the target to clear from non-target tissue of the subject prior to the step of irradiating.

27. A method of photodynamic therapy for treating hyperproliferative tissue in a subject, comprising:
(i) administering to the subject the conjugate of claim 1 or a pharmaceutically acceptable derivative thereof that preferentially associates with the hyperproliferative tissue, and
(ii) irradiating the subject with light of a wavelength and fluence sufficient to activate the conjugate, whereby the hyperproliferative tissue is destroyed or impaired.

28. A method for detecting the presence of a target tissue in a subject comprising:
(i) administering to the subject a sufficient quantity of the conjugate of claim 1 or a pharmaceutically acceptable derivative thereof that preferentially associates with the target tissue; and
(ii) visualizing the conjugate within the patient.

29. The method of claim 28 wherein the step of visualizing is accomplished by exposing the conjugate with light of sufficient energy to cause the composition to fluoresce.

30. A method for detecting a target in a biological sample, comprising:
(i) adding to the biological sample the conjugate of claim 1 or a pharmaceutically acceptable derivative thereof that binds to the target; and
(ii) detecting the composition.

31. The method of claim 32, wherein the biological sample is selected from the group consisting of blood, urine, saliva, tears, synovial fluid, sweat, interstitial fluid, sperm, cerebrospinal fluid, ascites fluid, and/or tumor tissue biopsy and circulating tumor cells.

32. A method of diagnosing an infecting agent in a patient, comprising:
(i) administering to the patient the conjugate of claim 1 or a pharmaceutically acceptable derivative thereof having a targeting moiety that binds to the infecting agent; and
(iii) visualizing the conjugate within the patient.

33. The method of claim 32 wherein the step of visualizing is accomplished by exposing the conjugate with light of sufficient energy to cause the conjugate to fluoresce.

34. A method of generating an image of a target tissue or target composition in a subject, comprising:
(i) administering to the subject the conjugate of claim 1 or a pharmaceutically acceptable derivative thereof; and
(ii) generating an image of at least a part of the subject to which the conjugate has preferentially associated.

35. A kit to treat hyperproliferative disorders, comprising the conjugate of claim 1 or a pharmaceutically acceptable derivative thereof and instructions teaching a method of photodynamic therapy.

36. A kit to specifically label a cell or tissue, comprising:
the conjugate of claim 1 or a pharmaceutically acceptable derivative thereof comprising a targeting moiety directed to the specific cell or tissue; and
instructions teaching a method of fluorescence imaging.

37. A combination, comprising:
the conjugate of claim 1 or a pharmaceutically acceptable derivative thereof; and
a light source.

38. A conjugate, comprising:
a tetrapyrrole or tetrapyrrole derivative photosensitizer that comprises a physiologically acceptable metal atom in its central coordination cavity;
a quenching agent comprising one or more suitable functional groups that coordinate to the axial position of the metal coordinated within the photosensitizer and position the quenching agent in an energy transfer conformation with the photosensitizer so that activation of the photosensitizer is quenched; and
a targeting moiety, wherein binding of the targeting moiety to a target disrupts the association of the axial ligand of the quenching agent to the metal, releasing the quenching agent and rendering the photosensitizer active.

39. The conjugate of claim 38, wherein the photosensitizer is a porphyrin.

40. The conjugate of claim 38, wherein the photosensitizer is a chlorin.

41. The conjugate of claim 38, wherein the photosensitizer is a bacteriochlorin.

42. The conjugate of claim 38, wherein the photosensitizer is Talaporfin sodium.

43. The conjugate of claim 38, wherein the quenching agent is □-carotene or a derivative thereof.

44. The conjugate of claim 38, wherein the targeting moiety is an antibody, and antibody fragment or other derivative of an antibody.

45. The conjugate of claim 38, wherein the targeting moiety is selected from the group consisting of an antigen, a ligand, a receptor, one member of a specific binding pair, a polyamide, a peptide, an oligosaccharide, a polysaccharide, a low density lipoprotein (LDL) or an apoprotein of LDL, a steroid, a steroid derivative, a hormone and a hormone-mimic.

46. The conjugate of claim 38, wherein the targeting moiety is dextran sulfate.

47. The conjugate of claim 38, wherein the quenching agent is 4-(4′-dimethylamino-phenylazo)benzoic acid (DABCYL) or 4-(4′-dimethylamino-phenylazo)sulfonic (DABSYL).

48. A pharmaceutical composition, comprising the conjugate of claim 38 or a pharmaceutically acceptable derivative thereof in a pharmaceutically acceptable carrier.

49. An article of manufacture, comprising:
packaging material; and
the conjugate of claim 38 or a pharmaceutically acceptable derivative thereof contained within the packaging material, wherein:
the conjugate or pharmaceutically acceptable derivative thereof is effective in a photodynamic therapy treatment for ameliorating the symptoms of a hyperproliferative disorder; and
the packaging material includes a label that indicates that the composition or salt thereof is used in a photodynamic therapy treatment for ameliorating the symptoms of a hyperproliferative disorder.

50. A method for administering a photodynamic therapy to a target, comprising:
(i) administering to a subject a conjugate of claim 38 or a pharmaceutically acceptable derivative thereof that preferentially associates with the target; and
(ii) irradiating the subject with light of a wavelength and total fluence sufficient to produce a therapeutic effect.

51. The method of claim 50, wherein the target is selected from the group consisting of a vascular endothelial tissue, a neovasculature tissue, a neovasculature tissue present in an eye, an abnormal vascular wall of a tumor, a solid tumor, a tumor of a head, a tumor of a neck, a tumor of an eye, a tumor of a gastrointestinal tract, a tumor of a liver, a tumor of a breast, a tumor of a prostate, a tumors of a lung, a nonsolid tumor, malignant cells of one of a hematopoietic tissue and a lymphoid tissue, lesions in a vascular system, a diseased bone marrow, and diseased cells in which the disease is one of an autoimmune and an inflammatory disease.

52. The method of claim 50, wherein the target composition is selected from the group consisting of bacteria, viruses, fungi, protozoa, and toxins.

53. The method of claim 50, further comprising the step of allowing sufficient time for any of the conjugate that is not preferentially associated to the target to clear from non-target tissue of the subject prior to the step of irradiating.

54. A method of photodynamic therapy for treating hyperproliferative tissue in a subject, comprising:
(i) administering to the subject the conjugate of claim 38 or a pharmaceutically acceptable derivative thereof that preferentially associates with the hyperproliferative tissue, and
(ii) irradiating the subject with light of a wavelength and fluence sufficient to activate the conjugate, whereby the hyperproliferative tissue is destroyed or impaired.

55. A method for detecting the presence of a target tissue in a subject comprising:
(i) administering to the subject a sufficient quantity of the conjugate of claim 38 or a pharmaceutically acceptable derivative thereof that preferentially associates with the target tissue; and
(ii) visualizing the conjugate within the patient.

56. The method of claim 55 wherein the step of visualizing is accomplished by exposing the conjugate with light of sufficient energy to cause the composition to fluoresce.

57. A method for detecting a target in a biological sample, comprising:
(i) adding to the biological sample the conjugate of claim 38 or a pharmaceutically acceptable derivative thereof that binds to the target; and
(ii) detecting the composition.

58. The method of claim 57, wherein the biological sample is selected from the group consisting of blood, urine, saliva, tears, synovial fluid, sweat, interstitial fluid, sperm, cerebrospinal fluid, ascites fluid, and/or tumor tissue biopsy and circulating tumor cells.

59. A method of diagnosing an infecting agent in a patient, comprising:
(i) administering to the patient the conjugate of claim 38 or a pharmaceutically acceptable derivative thereof having a targeting moiety that binds to the infecting agent; and
(iii) visualizing the conjugate within the patient.

60. The method of claim 59 wherein the step of visualizing is accomplished by exposing the conjugate with light of sufficient energy to cause the conjugate to fluoresce.

61. A method of generating an image of a target tissue or target composition in a subject, comprising:
(i) administering to the subject the conjugate of claim 38 or a pharmaceutically acceptable derivative thereof; and
(ii) generating an image of at least a part of the subject to which the conjugate has preferentially associated.

62. A kit to treat hyperproliferative disorders, comprising the conjugate of claim 38 or a pharmaceutically acceptable derivative thereof and instructions teaching a method of photodynamic therapy.

63. A kit to label specific a cell or tissue, comprising:
the conjugate of claim 38 or a pharmaceutically acceptable derivative thereof comprising a targeting moiety directed to the specific cell or tissue; and
instructions teaching a method of fluorescence imaging.

64. A combination, comprising:
the conjugate of claim 38 or a pharmaceutically acceptable derivative thereof; and
a light source.
